(19) United States
(12) Patent Application Publication
(10) Pub. No.: US 2008/0261973 A1
(43) Pub. Date: Oct. 23, 2008

(54) Sulphonamidoaniline Derivatives Being Janus Kinase Inhibitors

(75) Inventors: Hans-Georg Capraro, Rheinfelden (CH); Bernard Coupez, Colmar (FR); Pascal Furet, Thann (FR); Paul W. Manley, Arlesheim (CH); Carole Pissot Soldermann, Rosenau (FR)

Correspondence Address: NOVARTIS INSTITUTES FOR BIOMEDICAL RESEARCH, INC., 400 TECHNOLOGY SQUARE, CAMBRIDGE, MA, 02139, US

(73) Assignee: Novartis AG, Basel (CH)

(21) Appl. No.: 12/158,764

(22) PCT Filed: Dec. 20, 2006
(86) PCT No.: PCT/EP06/12311

§ 371 Date: Jun. 23, 2008

(30) Foreign Application Priority Data
Dec. 22, 2005 (GB) 0526246.4

(51) Int. Cl. C07D 473/32; C07D 487/04; A61K 31/52; A61K 31/5377

(52) U.S. Cl. 514/234.2; 544/277; 514/263.4; 514/263.2; 514/263.22; 544/118; 544/262; 514/262.1

(57) ABSTRACT

The invention relates to sulphonamidoanilines of formula I,

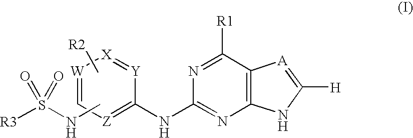

wherein A is N or CH, W, X, Y and Z are N or CH under the proviso that at least one of the three symbols W, X and Y represent CH, R¹ represents NR₄R₅ or OR₄, wherein R₄ represents optionally substituted alkyl, optionally substituted cycloalkyl optionally comprising one or two nitrogen or oxygen atoms, or substituted aryl, and R₅ represents hydrogen or unsubstituted or substituted alky, or R₄ and R₅ together with the nitrogen to which they are attached represent an optionally substituted five- or six-membered nitrogen containing monocyclic ring, an optionally substituted nitrogen containing fully saturated bicyclic ring, or an spirocyclic fully saturated ring system containing one or two nitrogen atoms, R² is hydrogen, lower alkenyl or alkyl, R³ is alkyl which is unsubstituted or mono-, di- or trisubstituted by halogen; alkenyl or aryl, and their salts; processes for their preparation, their application in the treatment of the human or animal body, the use thereof—alone or in combination with one or more other pharmaceutically active compounds—for the treatment of diseases, a method for the treatment of such a disease and the use of such a compound—alone or in combination with one or more other pharmaceutically active compounds—for the manufacture of a pharmaceutical preparation for the treatment of a proliferative disease.

The invention relates to new sulphonamidoaniline derivatives, processes for the preparation thereof, the application thereof in a process for the treatment of the human or animal body, the use thereof—alone or in combination with one or more other pharmaceutically active compounds—for the treatment especially of a proliferative disease, such as a tumor disease, a method for the treatment of such a disease and the use of such a compound—alone or in combination with one or more other pharmaceutically active compounds—for the manufacture of a pharmaceutical preparation (medicament) for the treatment of a proliferative disease.

Surprisingly, it has now been found that the sulphonamidoaniline derivatives of formula I, described below, have advantageous pharmacological properties and inhibit, for example, the tyrosine kinase activity of Janus kinases, such as JAK-2 kinase. Hence, the sulphonamidoaniline derivatives of formula I are suitable, for example, to be used in the treatment of diseases mediated by the tyrosine kinase activity of JAK-2 kinase, especially proliferative diseases such as tumor diseases, leukaemias, polycythemia vera, essential thrombocythemia, and myelofibrosis with myeloid metaplasia. Through the inhibition of JAK-3 kinase, compounds of the invention also have utility as immunosuppressive agents, for example for the treatment of diseases such as organ transplant rejection, lupus, multiple sclerosis, rheumatoid arthritis, psoriasis, dermatitis, Crohn's disease, type-1 diabetes and complications from type-1 diabetes.

The invention pertains to sulphonamidoanilines of formula I,

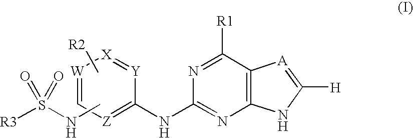

wherein
A is N or CH,
W, X, Y and Z are N or CH under the proviso that at least one of the three symbols W, X and Y represent CH,
R¹ represents NR₄R₅ or OR₄, wherein
   R₄ represents optionally substituted alkyl, optionally substituted cycloalkyl optionally comprising one or two nitrogen or oxygen atoms, or substituted aryl, and
   R₅ represents hydrogen or unsubstituted or substituted alkyl, or
   R₄ and R₅ together with the nitrogen to which they are attached represent an optionally substituted five- or six-membered nitrogen containing monocyclic ring, an optionally substituted nitrogen containing fully saturated bicyclic ring, or an spirocyclic fully saturated ring system containing one or two nitrogen atoms,
R² is hydrogen, lower alkenyl or alkyl,
R³ is alkyl which is unsubstituted or mono-, di- or trisubstituted by halogen; alkenyl or aryl, and to salts of such sulphonamidoanilines.

The invention pertains more specifically to sulphonamidoanilines of formula I, wherein
A is N or CH,
W, X, Y and Z are N or CH under the proviso that at least one of the three symbols W, X and Y represent CH,
R₁ represents NR₄R₅ or OR₄, wherein
   R₄ is selected from
   alkyl which is unsubstituted or substituted by hydroxy, lower alkoxy, amido, phenyl, amino-phenyl, di-(lower alkyl)amino-phenyl, trifluoromethyl phenyl, trifluoromethoxy phenyl, cyano phenyl, cyano lower alkyl phenyl, lower alkanoyl phenyl, lower alkanoyl amino-phenyl, lower alkanoyl (lower alkyl) amino-phenyl, lower alkyl sulfonylamino phenyl, lower alkoxy phenyl, hydroxy phenyl, hydroxy lower alkyl phenyl, 4-lower alkyl-piperazin-1-yl)-phenyl, nitro phenyl, imidazolyl, morpholinyl, di-(lower alkyl)amino, cyano, N-lower alkyl amino, C₅-C₇-cycloalkyl, benzo[1,2,5]oxadiazolyl, pyridyl or piperidinyl, or 1-aza-bicyclo[2.2.2]octyl, tetrahydrofuranyl, C₃-C₅-cycloalkyl being unsubstituted or substituted by lower alkyl or hydroxy; phenyl which is substituted by halogen; and
   R₅ represents hydrogen or lower alkyl being unsubstituted or substituted by hydroxyl or amino, or
   R₄ and R₅ together with the nitrogen atom to which they are attached represent morpholinyl, pyrrolidinyl which is unsubstituted or substituted by hydroxy lower alkyl or hydroxyl; piperazinyl substituted by pyridyl or lower alkyl; hexahydro-cyclopenta[b]pyrrol-1-yl which is unsubstituted or substituted by hydroxy lower alkyl; or diaza-spiro[5.5]undecyl,
R₂ is hydrogen, and
R₃ is lower alkyl, which is unsubstituted or mono-, di or trisubstituted by halogen, lower alkenyl, or phenyl monosubstituted by halogen.

In a specific embodiment, the present invention provides sulphonamidoaniline of formula I, wherein
A is N,
W, X, Y and Z are all CH,
R₁ represents NR₄R₅ or OR₄, wherein
R₄ represents isopropyl, 1,2,2-trimethyl-propyl, 2,2-dimethyl-propyl, 1,2-dimethyl-propyl, 1-ethyl-2,2-dimethyl-propyl, 2-hydroxy-1,1-dimethyl-ethyl, 1,2,2-trimethyl-butyl, 2-hydroxy-ethyl, 1-hydroxymethyl-2-methyl-propyl, 1-(2-hydroxy-ethyl)-2-methyl-propyl, 1-hydroxymethyl-2,2-dimethyl-propyl, 2-methoxy-ethyl, 2-isopropylamino-ethyl, 3-methyl-butyramide, benzyl, amino-benzyl, 3-dimethylamino-benzyl, 4-dimethylamino-benzyl, 3-acetylamino-benzyl, 3-(acetyl)-N-methylamino-benzyl, 3-cyano-benzyl, 4-cyanomethyl-benzyl, 3-acetyl-benzyl, methanesulfonylamino-benzyl, 3-(4-methyl-piperazin-1-yl)-benzyl, 3-trifluoromethyl-benzyl, 3-trifluoromethoxy-benzyl, 3-hydroxy-benzyl, 2-hydroxymethyl-benzyl, 2-hydroxyethyl-benzyl, 3-methoxy-benzyl, 3-nitro-benzyl, benzo[1,2,5]oxadiazol-5-ylmethyl, 1-phenyl-ethyl, 1-phenyl-propyl, 4-imidazolylethyl, 1H-Imidazol-2-ylmethyl, morpholin-4-yl-ethyl, diisopropylaminoethyl, dimethylaminoethyl, cyanoethyl, 2,3-dihydroxy-propyl, cyclohexylmethyl, 2-pyridin-2-yl-ethyl, pyridin-3-ylmethyl, piperidin-2-ylmethyl, 1-aza-bicyclo[2.2.2]oct-3-yl, tetrahydro-furan-3-yl, cyclopropyl, cyclobutyl, cyclopentyl, dimethyl-cyclopentyl, 2-hydroxy-cyclopentyl, 4-fluoro-phenyl, and
R₅ represents hydrogen, methyl, 2-amino-ethyl or 2-hydroxy-ethyl, or
R₄ and R₅ together with the nitrogen atom to which they are attached represent 2-hydroxymethyl-hexahydro-cyclopenta[b]pyrrol-1-yl, 1,4-diaza-spiro[5.5]undec-1-yl, 1,4-diaza-spiro[5.5]undec-4-yl, pyrrolidinyl which is unsubstituted or substituted by hydroxymethyl or hydroxy, piperazinyl substituted by 4-pyridyl or methyl, 4-methyl-imidazol-1-yl, morpholin-4-yl,
R₂ is hydrogen, and
R₃ is lower alkyl, which is unsubstituted or mono-, di or trisubstituted by fluoro, vinyl, or phenyl monosubstituted by fluoro.

The invention pertains in particular to sulphonamidoanilines of formula I, wherein
A is N or CH,
W, X, Y and Z are N or CH under the proviso that at least one of the three symbols W, X and Y represent CH,
R₁ represents NR₄R₅ or OR₄, wherein
   R₄ is selected from
   alkyl which is unsubstituted or substituted by hydroxy, lower alkoxy, phenyl, di-(lower alkyl)amino-phenyl, cyano lower alkyl phenyl, lower alkyl sulfonylamino phenyl, imidazolyl, morpholinyl, di-(lower alkyl)amino, cyano, C₅-C₇-cycloalkyl, pyridyl or piperidinyl, or 1-aza-bicyclo[2.2.2]octyl, tetrahydrofuranyl, cyclobutyl or cyclopentyl; and
   R₅ represents hydrogen or lower alkyl, or
   R₄ and R₅ together with the nitrogen atom to which they are attached represent morpholinyl, pyrrolidinyl which is unsubstituted or substituted by hydroxy lower alkyl or hydroxy, or piperazinyl substituted by pyridyl or lower alkyl,
R₂ is hydrogen, and
R₃ is lower alkyl, which is unsubstituted or mono-, di or trisubstituted by halogen, or phenyl monosubstituted by halogen,

and to salts of such sulphonamidoanilines.

Highly preferred are compounds, wherein the radicals have the following meanings:
A is N,
W, X, Y and Z are all CH,
R₁ represents NR₄R₅ or OR₄, wherein
   R₄ represents isopropyl, 1,2,2-trimethyl-propyl, 2-hydroxy-1,1-dimethyl-ethyl, 2-hydroxy-ethyl, 2-methoxy-ethyl,_benzyl, 3-dimethylamino-benzyl, 4-dimethylamino-benzyl, 4-cyanomethyl-benzyl, 4-methanesulfonylamino-benzyl, 1-phenyl-ethyl, 4-imidazolylethyl, 2-morpholin4-yl-ethyl, diisopropylaminoethyl, dimethylaminoethyl, cyanoethyl, 2,3-dihydroxy-propyl, cyclohexylmethyl, 2-pyridin-2-yl-ethyl, pyridin-3-ylmethyl, piperidin-2-ylmethyl, 1-aza-bicyclo[2.2.2]oct-3-yl, tetrahydro-furan-3-yl, cyclobutyl, cyclopentyl, and
   R₅ represents hydrogen or methyl, or
   R₄ and R₅ together with the nitrogen atom to which they are attached represent pyrrolidinyl which is unsubstituted or substituted by hydroxymethyl or hydroxy, piperazinyl substituted by 4-pyridyl or methyl, 4-methyl-imidazol-1-yl, morpholin4-yl,
R₂ is hydrogen, and
R₃ is lower alkyl, which is unsubstituted or mono-, di or trisubstituted by fluoro, or phenyl monosubstituted by fluoro,

and salts of such sulphonamidoanilines.

In formula I the following significances are preferred independently, collectively or in any combination or sub-combination:
(a) A is N,
(b) W, X, Y and Z all represent CH,
(c) R₁ is preferably NR₄R₅ wherein R₄ and R₅ have the meanings as defined herein,
(d) R₂ is preferably hydrogen,
(e) R₄ is preferably selected from isopropyl, 1,2,2-trimethyl-propyl, 2-hydroxy-1,1-dimethyl-ethyl, 2-hydroxy-ethyl, 2-methoxy-ethyl, benzyl, dimethylamino-benzyl, cyanomethyl-benzyl, methanesulfonylamino-benzyl, phenyl-ethyl, imidazolylethyl, morpholin-4-yl-ethyl, diisopropylaminoethyl, dimethylaminoethyl, cyanoethyl, 2,3-dihydroxy-propyl, cyclohexylmethyl, pyridyl-ethyl, pyridylmethyl, piperidylmethyl, 1-aza-bicyclo[2.2.2]octyl, tetrahydrofuranyl, cyclobutyl and cyclopentyl; or
R₄ and R₅ together with the nitrogen atom to which they are attached represent methyl-imidazolyl, morpholinyl, pyrrolidinyl which is unsubstituted or substituted by hydroxymethyl or hydroxy, or piperazinyl substituted by pyridyl or methyl,
(f) R₃ is lower alkyl, which is unsubstituted or mono-, di- or tri-substituted by fluoro, or phenyl monosubstituted by fluoro.

The general terms used hereinbefore and hereinafter preferably have within the context of this disclosure the following meanings, unless otherwise indicated:

The prefix “lower” denotes a radical having up to and including a maximum of 7, especially up to and including a maximum of 4 carbon atoms, the radicals in question being either linear or branched with single or multiple branching.

Where the plural form is used for compounds, salts, and the like, this is taken to mean also a single compound, salt, or the like.

Any asymmetric carbon atoms (for example in compounds of formula I, wherein R_{g} is lower alkyl) may be present in the (R)-, (S)- or (R,S)-configuration, preferably in the (R)- or (S)-configuration. The compounds may thus be present as mixtures of isomers or as pure isomers, preferably as enantiomer-pure diastereomers.

In the preferred embodiment, alkyl has up to a maximum of 12 carbon atoms and is especially lower alkyl.

Lower alkyl is preferably alkyl with from and including 1 up to and including 7, preferably from and including 1 to and including 4, and is linear or branched; preferably, lower alkyl is methyl, ethyl, butyl, such as n-butyl, sec-butyl, isobutyl, tert-butyl, propyl, such as n-propyl or isopropyl, 1,2,2-trimethyl-propyl, 2,2-dimethyl-propyl, 1,2-dimethyl-propyl, 1-ethyl-2,2-dimethyl-propyl, 1-ethyl-2-methyl-propyl, 1-methyl-2,2-dimethyl-propyl, 1,2,2-trimethyl-butyl.

Substituted alkyl denotes alkyl substituted by hydroxy, lower alkoxy, mono- or disubstituted amino, cyano, amido, C₅-C₇-cycloalkyl, five- or six-membered fully saturated heterocyclyl containing at least one nitrogen atom, five- or six-membered hetaryl containing at least one nitrogen atom, bicyclic hetaryl containing at least one nitrogen atom, or phenyl, which is unsubstituted or substituted by one or more, preferably up to three, especially one or two substituents, especially selected from amino, mono- or disubstituted amino, halogen, alkyl, substituted alkyl, hydroxy, esterified hydroxy, unsubstituted or substituted lower alkoxy, nitro, cyano, cyano lower alkyl, carboxy, esterified carboxy, alkanoyl, benzoyl, carbamoyl, N-mono- or N,N-disubstituted carbamoyl, amidino, guanidino, ureido, mercapto, sulfo, lower alkylthio, lower alkyl-piperazinyl, phenyl, phenoxy, phenylthio, phenyl-lower alkylthio, alkylphenylthio, lower alkylsulfinyl, phenylsulfinyl, phenyl-lower alkylsulfinyl, alkylphenylsulfinyl, lower alkane-sulfonyl, phenylsulfonyl, phenyl-lower alkylsulfonyl, alkylphenylsulfonyl, lower alkenyl, lower alkanoyl, C₅-C₇-cycloalkyl or lower alkylene dioxy bound at adjacent C-atoms of the ring, such as methylene dioxy; substituted alkyl as used in the definition of R⁴ especially denotes alkyl substituted by hydroxy, lower alkoxy, amido, phenyl, amino-phenyl, di-(lower alkyl)amino-phenyl, trifluoromethyl phenyl, trifluoromethoxy phenyl, cyano phenyl, cyano lower alkyl phenyl, lower alkanoyl phenyl, lower alkanoyl amino-phenyl, lower alkanoyl (lower alkyl) amino-phenyl, lower alkyl sulfonylamino phenyl, lower alkoxy phenyl, hydroxy phenyl, hydroxy lower alkyl phenyl, 4-lower alkyl-piperazin-1-yl)-phenyl, nitro phenyl, imidazolyl, morpholinyl, di-(lower alkyl)amino, cyano, N-lower alkyl amino, C₅-C₇-cycloalkyl, benzo[1,2,5]oxadiazolyl, pyridyl or piperidinyl.

Mono- or disubstituted amino is especially amino substituted by one or two radicals selected independently of one another from lower alkyl, such as methyl; hydroxy-lower alkyl, such as 2-hydroxyethyl; phenyl-lower alkyl; lower alkanoyl, such as acetyl; lower alkyl sulfonyl; benzoyl; substituted benzoyl, wherein the phenyl radical is especially substituted by one or more, preferably one or two, substituents selected from nitro, amino, halogen, N-lower alkylamino, N,N-di-lower alkylamino, hydroxy, cyano, carboxy, lower alkoxycarbonyl, lower alkanoyl, and carbamoyl; and phenyl-lower alkoxycarbonyl, wherein the phenyl radical is unsubstituted or especially substituted by one or more, preferably one or two, substituents selected from nitro, amino, halogen, N-lower alkylamino, N,N-di-lower alkylamino, hydroxy, cyano, carboxy, lower alkoxycarbonyl, lower alkanoyl, and carbamoyl; preferably substituted by one or two radicals selected independently of one another from lower alkyl, such as methyl; lower alkanoyl, such as acetyl and lower alkyl sulfonyl.

Esterified hydroxy is especially lower alkanoyloxy, benzoyloxy, lower alkoxycarbonyloxy, such as tert-butoxycarbonyloxy, or phenyl-lower alkoxycarbonyloxy, such as benzyloxycarbonyloxy.

Alkanoyl is primarily alkylcarbonyl, especially lower alkanoyl, e.g. acetyl.

Halogen is especially fluorine, chlorine, bromine, or iodine, especially fluorine, chlorine, or bromine.

Aryl is preferably phenyl or naphthyl, which in each case is unsubstituted or further substituted by up to 3 substituents preferably selected from amino, mono- or disubstituted amino, lower alkanoyl, cyano, nitro, halogen, especially fluoro, hydroxyl, alkoxy, which is unsubstituted or substituted by halo, or unsubstituted or substituted alkyl.

Haloaryl is preferably phenyl substituted by chloro or fluoro, preferably fluoro.

Cycloalkyl is especially C₃-C₆cycloalkyl, preferably cyclobutyl or cyclopentyl.

“Optionally substituted cycloalkyl optionally comprising one or two nitrogen or oxygen atoms” is especially C5-C7 cycloalkyl, tetrahydrofuranyl, piperidinyl, piperazinyl, morpholinyl, and pyrrolidinyl, being unsubstituted or substituted by lower alkyl or hydroxy.

“An optionally substituted nitrogen containing fully saturated bicyclic ring” is preferably a bicyclic fully saturated C₇-C₁₀carbocyclic ring system containing at least one nitrogen atom, being optionally substituted by hydroxy, lower alkyl, or hydroxy lower alkyl.

“An optionally substituted five- or six-membered nitrogen containing monocyclic ring” is preferably piperidinyl, piperazinyl or morpholinyl, being optionally substituted by hydroxy, lower alkyl, or hydroxy lower alkyl.

“A spirocyclic fully saturated ring system containing one or two nitrogen atoms” is preferably a spirocyclic fully saturated C₉-C₁₃carbocyclic ring system containing at least one nitrogen atom, being optionally substituted by hydroxy, lower alkyl, or hydroxy lower alkyl.

In view of the close relationship between the novel compounds in free form and those in the form of their salts, including those salts that can be used as intermediates, for example in the purification or identification of the novel compounds, any reference to the free compounds hereinbefore and hereinafter is to be understood as referring also to the corresponding salts, as appropriate and expedient.

For isolation or purification purposes it is also possible to use pharmaceutically unacceptable salts, for example picrates or perchlorates. For therapeutic use, only pharmaceutically acceptable salts or free compounds are employed (where applicable in the form of pharmaceutical preparations), and these are therefore preferred.

Such salts are formed, for example, as acid addition salts, preferably with organic or inorganic acids, from compounds of formula I with a basic nitrogen atom, especially the pharmaceutically acceptable salts. Suitable inorganic acids are, for example, halogen acids, such as hydrochloric acid, sulfuric acid, or phosphoric acid.

The compounds of formula I thereof have valuable pharmacological properties, as described hereinbefore and hereinafter.

The efficacy of the compounds of the invention as inhibitors of JAK-2-receptor tyrosine kinase activity can be demonstrated as follows:

Baculovirus including the amino acid domain ASP751-VAL1129 of the JAK-2 protein is obtainable by ProQinase, Freiburg, Germany. The virus is scaled up as following: Virus containing media is collected from the transfected cell culture and used for infection to increase its titer. Virus containing media obtained after two rounds of infection is used for large-scale protein expression. For large-scale protein expression 100 cm² round tissue culture plates are seeded with 5×10⁷ cells/plate and infected with 1 mL of virus-containing media (approx. 5 MOIs). After 3 days the cells are scraped off the plate and centrifuged at 500 rpm for 5 min. Cell pellets from 10-20, 100 cm² plates, are re-suspended in 50 mL of ice-cold lysis buffer (25 mMTris-HCl, pH7.5, 2 mMEDTA, 1%NP-40, 1 mM DTT, 1 mMP MSF).The cells are stirred on ice for 15 min and then centrifuged at 5000 rpms for 20 min. The protein is purified by loading the centrifuged cell lysate onto a 2 mL glutathione-sepharose column and washed three times with 10 mL of 25 mM Tris-HCl, pH 7.5, 2 mM EDTA, 1 mM DTT, 200 mM NaCl. The GST-tagged proteins are then eluted by 10 applications (1 mL each) of 25 mM Tris-HCl, pH 7.5, 10 mM reduced-glutathione, 100 mM NaCl, 1 mM DTT, 10% Glycerol and stored at −70° C.

The activity of JAK-2 is assayed in the presence or absence of inhibitor measuring the incorporation of 33P from [γ33P]ATP into appropriate substrates [Garcia-Echeverria C, Pearson M A, Marti A, et al (2004) In vivo antitumor activity of NVP-AEW541—A novel, potent, and selective inhibitor of the IGF-IR kinase. Cancer Cell; 5: 231-239]. The test compound is dissolved in DMSO (10 mM) and stored at −20° C. Serial dilutions are freshly made in DMSO and are 1000 times concentrated than test solutions (“pre-dilution plates”). They are further diluted with pure water to yield “master plates” containing 3 times concentrated test solutions in 3% DMSO. The final volume of the assay is 30 μL containing 10 μL of test solution (1% DMSO), 10 μL assay mix including the assay components described by Garcia-Echeverria (2004) and in the following section as well as 10 μL enzyme. The pipetting steps can be programmed to be performed either on the MultiPROBE lix, MultiPROBE IILx or HamiltonSTAR robots in the 96 well format.

The protein kinase assays are carried as described in details by Garcia-Echeverria (see above). The assay for JAK-2 is carried out in 96-well plates at ambient temperature for 10 min (filter-biding method) or 30 min (flash plates) in a finial volume of 30 μL including the following components: 300 ng of GST-JAK-2, 20 mM Tris-HCl, pH 7.5, 1.0 mM MnCl₂, 10 mM MgCl₂, 1 mM DTT, 3 μg/mL poly(Glu, Tyr) 4:1, 1% DMSO and 1.0 μM ATP (γ-[³³P]-ATP 0.1 μCi); The assays are terminated by the addition of 20 μl of 125 mM EDTA. The capturing of the phosphorylated peptides by the filter-binding method is performed as following: 40 μL of the reaction mixture are transferred onto Immobilon-PVDF membranes previously soaked for 5 min with methanol, rinsed with water, then soaked for 5 min with 0.5% H₃PO₄ and mounted on vacuum manifold with disconnected vacuum source. After spotting all samples, vacuum is connected and each well rinsed with 200 μl 0.5% H₃PO₄. Free membranes are removed and washed 4× on a shaker with 1.0% H3PO4, once with ethanol. Membranes are counted after drying at ambient temperature, mounting in Packard TopCount 96-well frame, and addition of 10 μl/well of Microscint. The plates are eventually sealed and counted in a microplate scintillation counter (TopCount NXT, TopCount NXT HTS, PerkinElmer, Brussels, Belgium).

The assays for the flash plate method is carried out in a total volume of 30 μL at RT in conventional 96-well flash plates. The reaction is stopped after 30 min by the addition of 20 μL of 125 mM EDTA The assay plates are then washed three times with PBS and dried at room temperature. The plates are sealed and counted in a microplate scintillation counter (TopCount NXT, TopCount NXT HTS). IC50 values are calculated by linear regression analysis of the percentage inhibition of the compound either in duplicate, at four concentrations (usually 0.01, 0.1, 1 and 10 μM) or as 8 single point IC50 starting at 10 μM following by 1:3 dilutions.

On the basis of these studies, a compound of formula I according to the invention shows therapeutic efficacy especially against disorders dependent on protein kinase, especially proliferative diseases mediated JAK-2 kinase activity.

The dosage of the active ingredient to be applied to a warm-blooded animal depends upon a variety of factors including type, species, age, weight, sex and medical condition of the patient; the severity of the condition to be treated; the route of administration; the renal and hepatic function of the patient; and the particular compound employed. A physician, clinician or veterinarian of ordinary skill can readily determine and prescribe the effective amount of the drug required to prevent, counter or arrest the progress of the condition. Optimal precision in achieving concentration of drug within the range that yields efficacy without toxicity requires a regimen based on the kinetics of the drug's availability to target sites. This involves a consideration of the distribution, equilibrium, and elimination of a drug. In general, a compound of formula I is applied in a daily dosage between about 1 mg and 1000 mg.

A compound of formula I can be administered alone or in combination with one or more other therapeutic agents, possible combination therapy taking the form of fixed combinations or the administration of a compound of the invention and one or more other therapeutic agents being staggered or given independently of one another, or the combined administration of fixed combinations and one or more other therapeutic agents. A compound of formula I can besides or in addition be administered especially for tumor therapy in combination with chemotherapy, radiotherapy, immunotherapy, surgical intervention, or a combination of these. Long-term therapy is equally possible as is adjuvant therapy in the context of other treatment strategies, as described above. Other possible treatments are therapy to maintain the patient's status after tumor regression, or even chemopreventive therapy, for example in patients at risk.

Therapeutic agents for possible combination are especially one or more antiproliferative, cytostatic or cytotoxic compounds, for example one or several agents selected from the group which includes, but is not limited to, an inhibitor of polyamine biosynthesis, an inhibitor of a protein kinase, especially of a serine/threonine protein kinase, such as protein kinase C, or of a tyrosine protein kinase, such as the EGF receptor tyrosine kinase, e.g. Iressa®, the VEGF receptor tyrosine kinase, e.g. PTK787 or Avastin®, or the PDGF receptor tyrosine kinase, e.g. STI571 (Glivec®)), a cytokine, a negative growth regulator, such as TGF-β or IFN-β, an aromatase inhibitor, e.g. letrozole (Femara®) or anastrozole, an inhibitor of the interaction of an SH2 domain with a phosphorylated protein, antiestrogens, topoisomerase I inhibitors, such as irinotecan, topoisomerase II inhibitors, microtubule active agents, e.g. paclitaxel or an epothilone, alkylating agents, antiproliferative antimetabolites, such as gemcitabine or capecitabine, platin compounds, such as carboplatin or cis-platin, bisphosphonates, e.g. AREDIA® or ZOMETA®, and monoclonal antibodies, e.g. against HER2, such as trastuzumab.

The structure of the active agents identified by code nos., generic or trade names may be taken from the actual edition of the standard compendium “The Merck Index” or from databases, e.g. Patents International (e.g. IMS World Publications). The corresponding content thereof is hereby incorporated by reference.

Furthermore, the invention relates to a method for the treatment of a proliferative disease which responds to an inhibition of the JAK-2-receptor tyrosine kinase activity, which comprises administering a compound of formula I or a pharmaceutically acceptable salt thereof, wherein the radicals and symbols have the meanings as defined above, in a quantity effective against the said disease, to a warm-blooded animal requiring such treatment.

The invention relates also to pharmaceutical compositions comprising an effective amount, especially an amount effective in the treatment of one of the above-mentioned disorders, of compound of the formula I or a pharmaceutically acceptable salt thereof together with pharmaceutically acceptable carriers that are suitable for topical, enteral, for example oral or rectal, or parenteral administration and that may be inorganic or organic, solid or liquid. Used for oral administration can be especially tablets or gelatin capsules that comprise the active ingredient together with diluents, for example lactose, dextrose, mannitol, and/or glycerol, and/or lubricants and/or polyethylene glycol. Tablets may also comprise binders, for example magnesium aluminum silicate, starches, such as corn, wheat or rice starch, gelatin, methylcellulose, sodium carboxymethylcellulose and/or polyvinylpyrrolidone, and, if desired, disintegrators, for example starches, agar, alginic acid or a salt thereof, such as sodium alginate, and/or effervescent mixtures, or adsorbents, dyes, flavorings and sweeteners. It is also possible to use the pharmacologically active compounds of the present invention in the form of parenterally administrable compositions or in the form of infusion solutions. The pharmaceutical compositions may be sterilized and/or may comprise excipients, for example preservatives, stabilisers, wetting agents and/or emulsifiers, solubilisers, salts for regulating the osmotic pressure and/or buffers. The present pharmaceutical compositions, which may, if desired, comprise other pharmacologically active substances are prepared in a manner known per se, for example by means of conventional mixing, granulating, confectioning, dissolving or lyophilising processes, and comprise approximately from 1% to 95%, especially from approximately 1% to approximately 20%, active ingredient(s).

A compound of the invention may be prepared by processes that, though not applied hitherto for the new compounds of the present invention, are known per se, especially a process characterized in that for the synthesis of a compound of the formula I wherein R₁ is NR₄R₅ in a first step a compound of formula II

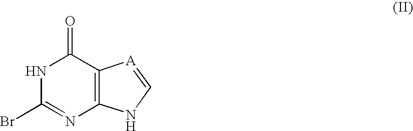

is reacted with an amine of formula III,

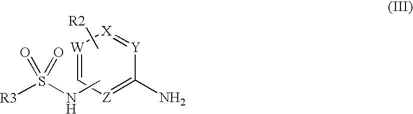

wherein the radicals and symbols have the meaning as defined for a compound of formula I above, under suitable reaction conditions, especially in a suitable alkanol at a temperature between 90° C. and 130° C. for a duration of 6 to 24 h, e.g. about 15 hours, providing a lactame of formula IV

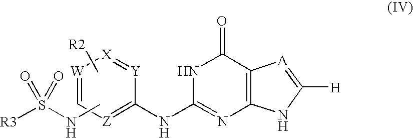

wherein the radicals and symbols have the meaning as defined for a compound of formula I above. Said lactame of formula IV is then in a second step transformed into the corresponding imidoyl chloride of formula V,

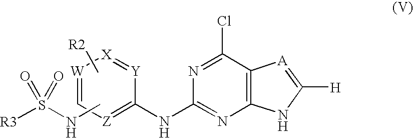

wherein the radicals and symbols have the meaning as defined for a compound of formula I above, by reaction with a suitable agent, such as phosporylchloride or thionylchloride.

The obtained imidoyl chloride of formula V is finally reacted with an amine of formula VI,

H—NR₄R₅ (VI)

under suitable conditions, e.g. heating a mixture containg the amine of formula VI and the imidoyl chloride of formula V for about 30 to 240 minutes to about 130 to 170° C., e.g. by applying microwave radiation, to provide a compound of the formula I wherein R₁ is NR₄R₅. Suitable processes and reaction conditions to obtain other derivatives of formula I are disclosed in the Examples.

### Protecting Groups

If one or more other functional groups, for example carboxy, hydroxy, amino, or mercapto, are or need to be protected in a compound of formulae II or III, because they should not take part in the reaction, these are such groups as are usually used in the sythesis of peptide compounds, and also of cephalosporins and penicillins, as well as nucleic acid derivatives and sugars.

The protecting groups may already be present in precursors and should protect the functional groups concerned against unwanted secondary reactions, such as acylations, etherifications, esterifications, oxidations, solvolysis, and similar reactions. It is a characteristic of protecting groups that they lend themselves readily, i.e. without undesired secondary reactions, to removal, typically by solvolysis, reduction, photolysis or also by enzyme activity, for example under conditions analogous to physiological conditions, and that they are not present in the end-products. The specialist knows, or can easily establish, which protecting groups are suitable with the reactions mentioned hereinabove and hereinafter.

The protection of such functional groups by such protecting groups, the protecting groups themselves, and their removal reactions are described for example in standard reference works, such as J. F. W. McOmie, “Protective Groups in Organic Chemistry”, Plenum Press, London and New York 1973, in T. W. Greene, “Protective Groups in Organic Synthesis”, Wiley, New York 1981, in “The Peptides”; Volume 3 (editors: E. Gross and J. Meienhofer), Academic Press, London and New York 1981, in “Methoden der organischen Chemie” (Methods of organic chemistry), Houben Weyl, 4th edition, Volume 15/l, Georg Thieme Verlag, Stuttgart 1974, in H.-D. Jakubke and H. Jescheit, “Aminosauren, Peptide, Proteine” (Amino acids, peptides, proteins), Verlag Chemie, Weinheim, Deerfield Beach, and Basel 1982, and in Jochen Lehmann, “Chemie der Kohlenhydrate: Monosaccharide und Derivate” (Chemistry of carbohydrates: monosaccharides and derivatives), Georg Thieme Verlag, Stuttgart 1974.

### Additional Process Steps

Salts of a compound of formula I with a salt-forming group may be prepared in a manner known per se. Acid addition salts of compounds of formula I may thus be obtained by treatment with an acid or with a suitable anion exchange reagent. A salt with two acid molecules (for example a dihalogenide of a compound of formula I) may also be converted into a salt with one acid molecule per compound (for example a monohalogenide); this may be done by heating to a melt, or for example by heating as a solid under a high vacuum at elevated temperature, for example from 130 to 170° C., one molecule of the acid being expelled per molecule of a compound of formula I.

Salts can usually be converted to free compounds, e.g. by treating with suitable basic agents, for example with alkali metal carbonates, alkali metal hydrogencarbonates, or alkali metal hydroxides, typically potassium carbonate or sodium hydroxide.

### Abbreviations:

AcOH acetic acid
DMSO dimethylsulfoxide
EtOAc ethyl acetate
EtOH ethanol
m.p. melting point
MS mass spectra
TLC thin layer chromatogram
Rₜ retention time
min minute(s)

The following Examples serve to illustrate the invention without limiting the invention in its scope. Temperatures are measured in degrees celsius (° C.). Unless otherwise indicated, the reactions take place at room temperature.

### EXAMPLES

### Example 1

### N-[3-(6-Cyclobutylamino-9H-purin-2-ylamino)-phenyl]-methanesulfonamide

Cyclobutylamine (63.9 μL, 0.74 mmol) is added to a stirred mixture of N-[3-(6-chloro-9H-purin-2-ylamino)-phenyl]-methanesulfonamide (Step 1.2, 169 mg, 0.5 mmol) and triethylamine (347 μL, 0.74 mmol) in EtOH (4 mL). The resulting solution is submitted to microwave irradiation for 90 min at 150° C. The solvent is evaporated off and the residue is dissolved in EtOAc, water is then added. The aqueous phase is separated and extracted with EtOAc. The combined organic layers are washed with water and brine, dried (Na₂SO₄) and concentrated to give a residue, which is purified by column chromatography (SiO₂; CH₂Cl₂/EtOH/NH₃ 90:9:1) to afford the title compound as a pale pink solid, m.p. 142-146° C.; MS: 374 (M+1)⁺.

The starting material is prepared as follows:

Step 1.1: N-[3-(6-Oxo-6.9-dihydro-1H-purin-2-ylamino)-phenyl]-methanesulfonamide

N-(3-Aminophenyl)methanesulfonamide (4.1 g, 22 mmol) is added to a solution of 2-bromo-1,9-dihydro-purin-6-one (4.30 g, 20 mmol) in 2-methoxythoxyethanol (120 mL). The resulting solution is heated at 110° C. for 15 h. The mixture is cooled to room temperature and the solvent is evaporated off under reduced pressure. Water is then added and the suspension is filtered, washed twice with water to give, after drying in high vacuum, the title compound as a pale pink solid, MS: 321 (M+1)⁺.

Step 1.2: N-[3-(6-Chloro-9H-purin-2-ylamino)-phenyl]-methanesulfonamide

N-[3-(6-Oxo-6,9-dihydro-1H-purin-2-ylamino)-phenyl]-methanesulfonamide (step 1.1, 1.6 g, 5 mmol) is added to phosphorousoxychloride (40 mL) and heated at 110° C. for 40 h. After cooling, the remaining POCI₃ is evaporated off under reduced pressure to afford the title compound as a brown solid, which is used directly without further purification, MS: 439 (M+1)⁺.

### Example 2

### N-[3-(6-Isopropylamino-9H-purin-2-ylamino)-phenyl]-methanesulfonamide

This compound can be obtained analogously to Example 1, utilising isopropylamine in lieu of cyclobutylamine, to afford the title compound as a colorless solid, m.p. 161-165° C., MS: 362 (M+1)⁺

### Example 3

### N-{3-[6-((S)-2-Hydroxymethyl-pyrrolidin-1-yl)-9H-purin-2-ylamino]-phenyl}-methanesulfonamide

This compound can be obtained analogously to Example 1, utilising S-2-(hydroxymethyl)-pyrrolidine in lieu of cyclobutylamine, to afford the title compound as a pale pink solid, m.p. 192-197° C., MS: 404 (M+1)⁺

### Example 4

### N-(3-{6-[2-(1H-Imidazol-4-yl)-ethylamino]-9H-purin-2-ylamino]-phenyl}-methanesulfonamide

This compound can be obtained analogously to Example 1, utilising 2-(1H-Imidazol-4-yl)ethylamine in lieu of cyclobutylamine, to afford the title compound as a pale pink solid, m.p. 205-210° C., MS: 414 (M+1)⁺

### Example 5

### N-(3-{6-[2-Diisopropylamino-ethylamino]-9H-purin-2-ylamino]-phenyl}-methanesulfonamide

This compound can be obtained analogously to Example 1, utilising 2-diisopropylaminoethylamine in lieu of cyclobutylamine, to afford the title compound as a pale pink solid, m.p. 226-230° C., MS: 447 (M+1)⁺.

### Example 6

### N-(3-{6-[2-Dimethylamino-ethylamino]-9H-purin-2-ylamino]-phenyl}-methanesulfonamide

This compound can be obtained analogously to Example 1, utilising 2-dimethylaminoethylamine in lieu of cyclobutylamine, to afford the title compound as a beige solid, m.p. 191-195° C., MS: 391 (M+1)⁺.

### Example 7

### N-{3-[6-(4-Pyridin-4-yl-piperazin-1-yl)-9H-purin-2-ylamino]-phenyl}-methanesulfonamide

This compound can be obtained analogously to Example 1, utilising 1-(4-pyridyl)piperazine in lieu of cyclobutylamine, to afford the title compound as a beige solid, m.p. 227-230° C., MS: 466 (M+1)⁺.

### Example 8

### N-(3-{6-[(2-Cyano-ethyl)-methyl-amino]-9H-purin-2-ylamino}-phenyl)-methanesulfonamide

This compound can be obtained analogously to Example 1, utilising 3-methylaminopropionitrile in lieu of cyclobutylamine, to afford the title compound as a beige solid, m.p. 218-222° C., MS: 387 (M+1)⁺

### Example 9

### N-{3-[6-(1-Aza-bicyclo[2.2.2]oct-3-ylamino)-9H-purin-2-ylamino]-phenyl}-methanesulfonamide

This compound can be obtained analogously to Example 1, utilising 3-aminoquinuclidine dihydrochloride in lieu of cyclobutylamine, to afford the title compound as a beige solid, m.p. 200-204° C., MS: 429 (M+1)⁺

### Example 10

### N-{3-[6-(2-Pyridin-2-yl-ethylamino)-9H-purin-2-ylamino]-phenyl}-methanesulfonamide

This compound can be obtained analogously to Example 1, utilising 2-(2-aminoethyl)pyridine in lieu of cyclobutylamine, to afford the title compound as a colorless solid, m.p. 199-202° C., MS: 425 (M+1)⁺.

### Example 11

### N-{3-[6-(2,3-Dihydroxy-propylamino)-9H-purin-2-ylamino]-phenyl}-methanesulfonamide

This compound can be obtained analogously to Example 1, utilising 3-amino-1,2 propanediol in lieu of cyclobutylamine, to afford the title compound as a grey solid, m.p. 164-168° C., . MS: 394 (M+1)⁺.

### Example 12

### N-{3-[6-(3-Hydroxy-pyrrolidin-1-yl)-9H-purin-2-ylamino]-phenyl}-methanesulfonamide

This compound can be obtained analogously to Example 1, utilising 3-pyrrolidinol in lieu of cyclobutylamine, to afford the title compound as a colorless solid, m.p. 243-248° C., MS: 390 (M+1)⁺.

### Example 13

### N-{3-[6-(4-Methyl-imidazol-1-yl)-9H-purin-2-ylamino]-phenyl}-methanesulfonamide

This compound can be obtained analogously to Example 1, utilising 4-(5)-methylimidazole in lieu of cyclobutylamine, to afford the title compound as a colorless solid, m.p. 298-301° C., MS: 385 (M+1)⁺.

### Example 14

### N-[3-(6-Pyrrolidin-1-yl-9H-purin-2-ylamino)-phenyl]-methanesulfonamide

This compound can be obtained analogously to Example 1, utilising pyrrolidine in lieu of cyclobutylamine, to afford the title compound as a colorless solid, m.p. 164-168° C., MS: 374 (M+1)⁺

### Example 15

### N-(3-{6-[(Pyridin-3-ylmethyl)-amino]-9H-purin-2-ylamino}-phenyl)-methanesulfonamide

This compound can be obtained analogously to Example 1, utilising 3-pyridylmethylamine in lieu of cyclobutylamine, to afford the title compound as a colorless solid, m.p. 144-147° C., MS: 411 (M+1)⁺.

### Example 16

### N-{3-[6-(2-Hydroxy-1,1-dimethyl-ethylamino)-9H-purin-2-ylamino]-phenyl}-methanesulfonamide

This compound can be obtained analogously to Example 1, utilising 2-amino-2-methyl-1-propanol in lieu of cyclobutylamine, to afford the title compound as a colorless solid, m.p. 215-218° C., MS: 392 (M+1)⁺.

### Example 17

### N-{3-[6-(2-Hydroxy-ethylamino)-9H-purin-2-ylamino]-phenyl}-methanesulfonamide

This compound can be obtained analogously to Example 1, utilising 2-aminoethanol in lieu of cyclobutylamine, to afford the title compound as a colorless solid, m.p. 163-166° C., MS: 364 (M+1)⁺.

### Example 18

### N-{3-[6-(4-Methyl-piperazin-1-yl)-9H-purin-2-ylamino]-phenyl}-methanesulfonamide

This compound can be obtained analogously to Example 1, utilising N-methylpiperazine in lieu of cyclobutylamine, to afford the title compound as a colorless solid, m.p. 148-151° C., MS: 403 (M+1)⁺

### Example 19

### N-[3-(6Morpholin-4-yl-9H-purin-2-ylamino)-phenyl]-methanesulfonamide

This compound can be obtained analogously to Example 1, utilising morpholine in lieu of cyclobutylamine, to afford the title compound as a pale pink solid, Rₜ=0.659 min (Acquity UPLC BEH C18, 2.1×50 mm, 1.7 micron, detection 215 nM, 0.1 min 2% CH₃CN in H₂O, 2% to 100% CH₃CN in H₂O in 1.5 min, 0.4 min 100% CH₃CN+0.1% TFA, flow rate 1.0 ml/min); MS: 390 (M+1)⁺.

### Example 20

### N-{3-[6-(4-Dimethylamino-benzylamino)-9H-purin-2-ylamino]-phenyl}-methanesulfonamide

This compound can be obtained analogously to Example 1, utilising 4-dimethylamino-benzylamine in lieu of cyclobutylamine, to afford the title compound as a pale pink solid, m.p. 138-142° C., MS: 453 (M+1)⁺.

### Example 21

### N-(3-{6-[(Piperidin-2-ylmethyl)-amino]-9H-purin-2-ylamino}-phenyl)-methanesulfonamide

This compound can be obtained analogously to Example 1, utilising 2-(aminomethyl)piperidine in lieu of cyclobutylamine, to afford the title compound as a colorless solid, m.p. 147-150° C., MS: 417 (M+1)⁺.

### Example 22

### N-{3-[6-(2-Morpholin-4-yl-ethylamino)-9H-purin-2-ylamino]-phenyl}-methanesulfonamide

This compound can be obtained analogously to Example 1, utilising 4(2-aminoethyl)-morpholine in lieu of cyclobutylamine, to afford the title compound as a pale pink solid, m.p. 233-237° C., MS: 433 (M+1)⁺.

### Example 23

### N-[3-(6-Cyclopentyloxy-9H-purin-2-ylamino)-phenyl]-methanesulfonamide

Sodium (20 mg, 0.88 mmol) is dissolved in cyclopentanol (814 μL, 8.86 mmol) at 90° C. N-[3-(6-chloro-9H-purin-2-ylamino)-phenyl]-methanesulfonamide (Step 1.2, 100 mg, 0.3 mmol) is then added to the stirred solution and heating is continued at 90° C. for 10 h. The mixture is cooled to room temperature, neutralized (AcOH), and the solvent is evaporated off under reduced pressure. The resulting residue is dissolved in EtOAc, washed with water and with brine, dried (Na₂SO₄) and the solvent is evaporated off under reduced pressure to give a residue, which is purified by column chromatography (SiO₂; CH₂Cl₂/EtOH/NH₃ 95:5:0.5 to 90:9:1) to afford the title compound as a colorless solid, m.p. 137-140° C., MS: 389 (M+1)⁺.

### Example 24

### N-[3-(6-Cyclohexylmethoxy-9H-purin-2-ylamino)-phenyl]-methanesulfonamide

This compound can be obtained analogously to Example 23, utilising cyclohexylmethanol in lieu of cyclopentanol, to afford the title compound as a colorless solid, m.p. 169-172° C., MS: 417 (M+1)⁺

### Example 25

### N-{3-[6-(Tetrahydro-furan-3-yloxy)-9H-purin-2-ylamino]-phenyl}-methanesulfonamide

This compound can be obtained analogously to Example 23, utilising 3-hydroxytetra-hydrofurane in lieu of cyclopentanol, to afford the title compound as a colorless solid, m.p. 255-257° C., MS: 391 (M+1)⁺

### Example 26

### N-[3-(6-Benzyloxy-9H-purin-2-ylamino)-phenyl]-methanesulfonamide

Benzylalcohol (63 μL, 0.6 mmol) is added to a stirred suspension of NaH (9 mg, 0.2 mmol) in DMSO (3 mL). After 1 h at room temperature, a solution of 1-[2-(3-methanesulfonylamino-phenylamino)-9H-purin-6-yl]-4-aza-1-azonium-bicyclo[2.2.2]octane chloride (Step 26.1, 50 mg, 0.1 mmol) in DMSO (2 mL) is added to solution. The resulting solution is then further stirred for 72 h and poured onto water (15 mL). The suspension is filtered, washed with water to give the title compound as a colorless solid, Rₜ=0.828 min (Acquity UPLC BEH C18, 2.1×50 mm, 1.7 micron, detection 215 nM, 0.1 min 2% CH₃CN in H₂O, 2% to 100% CH₃CN in H₂O in 1.5 min, 0.4 min 100% CH₃CN+0.1% TFA, flow rate 1.0 ml/min); MS: 411 (M+1)⁺

The starting material is prepared as follows:

Step 26.1: 1-[2-(3-Methanesulfonylamino-phenylamino)-9H-purin-6-yl]-4-aza-1-azonium-bicyclo[2.2.2]octane chloride

1,4-diazabicyclo[2.2.2]octane (182 mg, 1.62 mmol) is added to a suspension of -[3-(6-chloro-9H-purin-2-ylamino)-phenyl]-methanesulfonamide (Step 1.2, 100 mg, 0.3 mmol) in EtOH (25 mL). The mixture is stirred at room temperature for 72 h. The resulting suspension is filtered, washed twice with EtOH to give, the title compound as a beige solid, which is used directly without further purification.

### Example 27

### N-[3-(6-Cyclopentylamino-9H-purin-2-ylamino)-phenyl]-C,C,C-trifluoro-methanesulfonamide

This compound can be obtained analogously to Example 1, utilising cyclopentylamine in lieu of cyclobutylamine and N-[3-(6-chloro-9H-purin-2-ylamino)-phenyl]-C,C,C-trifluoro-methanesulfonamide (Step 26.4) in lieu of N-[3-(6-Chloro-9H-purin-2-ylamino)-phenyl]-methanesulfonamide (step 1.2), to afford the title compound as a beige solid, m.p. 243-246° C., MS: 442 (M+1)⁺

The starting material is prepared as follows:

Step 27.1: C,C,C-Trifluoro-N-(3-nitro-phenyl)-methanesulfonamide

Trifluoromethanesulfonic anhydride (18.1 mL, 106 mmol) is added to a solution of 3-nitroaniline (15 g, 106 mmol) and triethylamine (14.9 ml, 106 mmol) in chloroform (275 mL) at 0° C. The cooling bath is removed and the mixture is heated at reflux for 1 h. After cooling to room temperature, the reaction mixture is poured onto a 10% NaOH solution. The aqueous phase is separated and washed with CHCl₃. The basic aqueous phase is acidified with concentrated HCl and extracted with EtOAc. The combined organic layers are washed with saturated brine, dried (MgSO₄) and the solvent is evaporated off under reduced pressure to afford the title compound as a yellow solid.

### Step 27.2 N-(3-Amino-phenyl)-C,C,C-trifluoro-methanesulfonamide

A solution of C,C,C-trifluoro-N-(3-nitro-phenyl)-methanesulfonamide (20.8 g, 76 mmol) in EtOH (500 mL) is hydrogenated at room temperature over Pd on charcoal. After 1 h, the suspension is filtered off (hyflo) and the solvent is evaporated off under reduced pressure, to afford the title compound as a pale brown solid.

Step 27.3: C,C,C-Trifluoro-N-[3-(6-oxo-6,9-dihydro-1H-purin-2-ylamino)-phenyl]-methanesulfonamide

This compound can be obtained analogously to Example 1.1 utilising N-(3-amino-phenyl)-C,C,C-trifluoro-methanesulfonamide in lieu of N-(3-aminophenyl)methanesulfonamide, to afford the title compound as a beige solid, MS: 375 (M+1)⁺

Step 27.4: N-[3-(6-Chloro-9H-purin-2-ylamino)-phenyl]-C,C,C-trifluoro-methanesulfonamide

This compound can be obtained analogously to Example 1.2 utilising C,C,C-trifluoro-N-[3-(6-oxo-6,9-dihydro-1H-purin-2-ylamino)-phenyl]-methanesulfonamide in lieu of N-[3-(6-oxo-6,9-dihydro-1H-purin-2-ylamino)-phenyl]-methanesulfonamide (Step 1.1), to afford the title compound as a beige solid which is used directly without further purification.

### Example 28

### Ethanesulfonic acid [3-(6-cyclopentylamino-9H-purin-2-ylamino)-phenyl]-amide

This compound can be obtained analogously to Example 27, utilising ethanesulfonic acid [3-(6-chloro-9H-purin-2-ylamino)-phenyl]-amide in lieu of N-[3-(6-chloro-9H-purin-2-ylamino)-phenyl]-C,C,C-trifluoro-methanesulfonamide (Step 27.4), to afford the title compound as a beige solid, Rₜ=0.821 min (Acquity UPLC BEH C18, 2.1×50 mm, 1.7 micron, detection 215 nM, 0.1 min 2% CH₃CN in H₂O, 2% to 100% CH₃CN in H₂O in 1.5 min, 0.4 min 100% CH₃CN+0.1% TFA, flow rate 1.0 ml/min); MS: 402 (M+1)⁺.

### Example 29

### Propane-1-sulfonic acid [3-(6-cyclopentylamino-9H-purin-2-ylamino)-phenyl]-amide

This compound can be obtained analogously to Example 27, utilising propane-1-sulfonic acid [3-(6-chloro-9H-purin-2-ylamino)-phenyl]-amide in lieu of N-[3-(6-chloro-9H-purin-2-ylamino)-phenyl]-C,C,C-trifluoro-methanesulfonamide (Step 27.4), to afford the title compound as a beige solid, Rₜ=0.875 min (Acquity UPLC BEH C18, 2.1×50 mm, 1.7 micron, detection 215 nM, 0.1 min 2% CH₃CN in H₂O, 2% to 100% CH₃CN in H₂O in 1.5 min, 0.4 min 100% CH₃CN+0.1% TFA, flow rate 1.0 ml/min); MS: 416 (M+1)⁺.

### Example 30

### Propane-2-sulfonic acid [3-(6-cyclopentylamino-9H-purin-2-ylamino)-phenyl]-amide

This compound can be obtained analogously to Example 27, utilising propane-2-sulfonic acid [3-(6-chloro-9H-purin-2-ylamino)-phenyl]-amide in lieu of N-[3-(6-chloro-9H-purin-2-ylamino)-phenyl]-C,C,C-trifluoro-methanesulfonamide (Step 27.4), to afford the title compound as a brown solid, Rₜ=0.862 min (Acquity UPLC BEH C18, 2.1×50 mm, 1.7 micron, detection 215 nM, 0.1 min 2% CH₃CN in H₂O, 2% to 100% CH₃CN in H₂O in 1.5 min, 0.4 min 100% CH₃CN+0.1% TFA, flow rate 1.0 ml/min); MS: 416 (M+1)⁺.

### Example 31

### N-{3-[6-(3-Dimethylamino-benzylamino)-9H-purin-2-ylamino]-phenyl}-methanesulfonamide

This compound can be obtained analogously to Example 1, utilising 3-dimethylamino-benzylamine in lieu of cyclobutylamine, to afford the title compound as a beige solid, m.p. 148-151° C., flow rate 1 ml/min at 25 or 30° C.); MS: 453 (M+1)⁺.

### Example 32

### N-{3-[6-(2-Methoxy-ethoxy)-9H-purin-2-ylamino]-phenyl}-methanesulfonamide

This compound can be obtained analogously to Example 23, utilising 2-methoxyethanol in lieu of cyclopentanol, to afford the title compound as a light yellow solid, m.p. 201-204° C., MS: 379 (M+1)⁺.

### Example 33

### N-{3-[6-(4-Cyanomethyl-benzylamino)-9H-purin-2-ylamino]-phenyl}-methanesulfonamide

This compound can be obtained analogously to Example 1, utilising (4-Aminomethyl-phenyl)-acetonitrile in lieu of cyclobutylamine, to afford the title compound as a colorless solid.

### Example 34

### N-{3-[6-(4-Methanesulfonylamino-benzylamino)-9H-purin-2-ylamino]-phenyl}-methanesulfonamide

This compound can be obtained analogously to Example 1, utilising N-(4-Aminomethyl-phenyl)-methanesulfonamide in lieu of cyclobutylamine, to afford the title compound as a colorless oil.

### Example 35

### N-{3-[6-(4-Cyanomethyl-benzylamino)-9H-purin-2-ylamino]-phenyl}-4-fluoro-benzenesulfonamide

This compound can be obtained analogously to Example 1, utilising (4-Aminomethyl-phenyl)-acetonitrile in lieu of cyclobutylamine and N-[3-(6-Chloro-9H-purin-2-ylamino)-phenyl]-4-fluoro-benzenesulfonamide in lieu of N-[3-(6-Chloro-9H-purin-2-ylamino)-phenyl]-methanesulfonamide (step 1.2), to afford the title compound as a colorless solid.

### Example 36

### N-{3-[6-(Isopropyl-methyl-amino)-9H-purin-2-ylamino]-phenyl}-methanesulfonamide

This compound can be obtained analogously to Example 1, utilising Isopropyl-methyl-amine in lieu of cyclobutylamine, to afford the title compound as a light pink solid, m.p. 144-147° C., MS: 376 (M+1)⁺

### Example 37

### N-[3-(6-Cyclopentylamino-9H-purin-2-ylamino)-phenyl]-methanesulfonamide

This compound can be obtained analogously to Example 1, utilising cyclopentylamine in lieu of cyclobutylamine, to afford the title compound as a light beige solid, m.p. 234-239° C., MS: 388 (M+1)⁺.

### Example 38

### N-{3-[6-((S)-1,2,2-Trimethyl-propylamino)-9H-purin-2-ylamino]-phenyl}-methanesulfonamide

This compound can be obtained analogously to Example 1, utilising (S)-1,2,2-Trimethyl-propylamine in lieu of cyclobutylamine, to afford the title compound as a light beige solid, m.p. 150-153° C., MS: 404 (M+1)⁺

### Example 39

### N-{3-[6-((R)-1,2,2-Trimethyl-propylamino)-9H-purin-2-ylamino]-phenyl}-methanesulfonamide

This compound can be obtained analogously to Example 1, utilising (R)-1,2,2-Trimethyl-propylamine in lieu of cyclobutylamine, to afford the title compound as a light beige solid, m.p. 169-171° C., MS: 404 (M+1)⁺.

### Example 40

### N-{3-[6-((S)-1-Phenyl-ethylamino)-9H-purin-2-ylamino]-phenyl}-methanesulfonamide

This compound can be obtained analogously to Example 1, utilising (S)-1-Phenyl-ethylamine in lieu of cyclobutylamine, to afford the title compound as a light beige solid, m.p. 141-144° C., MS: 424 (M+1)⁺

### Example 41

### N-{3-[6-((R)-1-Phenyl-ethylamino)-9H-purin-2-ylamino]-phenyl}-methanesulfonamide

This compound can be obtained analogously to Example 1, utilising (R)-1-Phenyl-ethylamine in lieu of cyclobutylamine, to afford the title compound as a light beige solid, m.p. 136-139° C., MS: 424 (M+1)⁺

### Example 42

### N-[3-(4-Cyclopentylamino-7H-pyrrolo[2,3-d]pyrimidin-2-ylamino)-phenyl]-methanesulfonamide

This compound can be obtained analogously to Example 1, utilising cyclopentylamine in lieu of cyclobutylamine and N-[3-(4-Chloro-7H-pyrrolo[2,3-d]pyrimidin-2-ylamino)-phenyl]-methanesulfonamide step 42.3) in lieu of N-[3-(6-chloro-9H-purin-2-ylamino)-phenyl]-methanesulfonamide to afford the title compound as a light beige solid, Rₜ=0.884 min (Acquity UPLC BEH C18, 2.1×50 mm, 1.7 micron, detection 215 nM, 0.1 min 2% CH₃CN in H₂O, 2% to 100% CH₃CN in H₂O in 1.5 min, 0.4 min 100% CH₃CN+0.1% TFA, flow rate 1.0 ml/min); MS: 387 (M+1)⁺

The starting material is prepared as follows:

Step 42.1 N-[3-(4-Amino-6-hydroxy-pyrimidin-2-ylamino)-phenyl]-methanesulfonamide

N-(3-Aminophenyl)methanesulfonamide (7.4 g, 40 mmol) is added to a solution of 6-Amino-2-bromo-pyrimidin-4-ol (7.6 g, 40 mmol) (described in Hirayama et al., Chemical & Pharmaceutical Bulletin (1976), 24(1), 26-35) in 2-methoxythoxyethanol (240 mL). The resulting solution is heated at 110° C. for 2 h. The mixture is cooled to room temperature and the solvent is evaporated off under reduced pressure. Water is then added and the suspension is filtered, washed twice with water to give, after drying in high vacuum, the title compound as a grey solid.

Step 42.2: N-[3-(4-Oxo-4,7-dihydro-3H-pyrrolo[2,3-d]pyrimidin-2-ylamino)-phenyl]-methanesulfonamide

Sodium acetate ( 344 mg, 4.2 mmol) is added to a solution of N-[3-(4-Amino-6-hydroxy-pyrimidin-2-ylamino)-phenyl]-methanesulfonamide (590 mg, 2 mmol) in a mixture of CH₃CN (25 mL) and H₂O (12 mL). A solution of chloroacetaldehyde (258 μL, 2 mmol) in CH₃CN (5 mL) and H₂O (3 mL) is then added and the reaction mixture is heated at 50° C. for 7 h. The mixture is cooled to room temperature and the solvent is evaporated off under reduced pressure. Water is then added and the suspension is filtered, washed twice with water to give, after drying in high vacuum, the title compound as a beige solid.

Step 42.3: N-[3-(4-Chloro-7H-pyrrolo[2,3-d]pyrimidin-2-ylamino)-phenyl]-methanesulfonamide

N-[3-(4-Oxo-4,7-dihydro-3H-pyrrolo[2,3-d]pyrimidin-2-ylamino)-phenyl]-methanesulfonamide (step 40.2, 320 mg, 1 mmol) is added to phosphorousoxychloride (20 mL) and heated at 110° C. for 4 h. After cooling, the remaining POCI₃ is evaporated off under reduced pressure to afford the title compound as a brown solid, which is used directly without further purification.

### Example 43

### N-[3-(4-Cyclopentyloxy-7H-pyrrolo[2,3-d]pyrimidin-2-ylamino)-phenyl]-methanesulfonamide

This compound can be obtained analogously to Example 23, utilising N-[3-(4-Chloro-7H-pyrrolo[2,3-d]pyrimidin-2-ylamino)-phenyl]-methanesulfonamide (step 42.3) in lieu of N-[3-(6-chloro-9H-purin-2-ylamino)-phenyl]-methanesulfonamide to afford the title compound as a colorless solid, m.p. 203-205° C., MS: 388 (M+1)⁺

### Example 44

### N-[4-(6-Cyclopentylamino-9H-purin-2-ylamino)-phenyl]-methanesulfonamide

This compound can be obtained analogously to Example 27, utilising N-[4-(6-Chloro-9H-purin-2-ylamino)-phenyl]-methanesulfonamide in lieu of N-[3-(6-chloro-9H-purin-2-ylamino)-phenyl]-C,C,C-trifluoro-methanesulfonamide (step 26.4), to afford the title compound as a beige solid, m.p. 261-265° C., MS: 388 (M+1)⁺

### Example 45

### N-(3-{6-[(1H-Imidazol-2-ylmethyl)-amino]-9H-purin-2-ylamino}-phenyl)-methanesulfonamide

This compound can be obtained analogously to Example 1, utilising C-(1H-Imidazol-2-yl)-methylamine hydrochloride in lieu of cyclobutylamine, to afford the title compound as a colorless solid, m.p. 216-220° C., MS: 400 (M+1)⁺.

### Example 46

### N-{3-[6-(4-Fluoro-phenoxy)-9H-purin-2-ylamino]-phenyl}-methanesulfonamide

4-fluorophenol (225 g, 2 mmol) is added to a stirred suspension of NaH (60 mg, 1.5 mmol) in THF (3 mL). After 30 min. at room temperature, a solution of N-[3-(6-chloro-9H-purin-2-ylamino)-phenyl]-methanesulfonamide (Step 1.2, 169 mg, 0.5 mmol) in THF (2 mL) is added to solution. The resulting solution is then heated at 60° C. for 16h. After cooling, the solvent is evaporated off and the residue is dissolved in EtOAc, water is then added. The aqueous phase is separated and extracted with EtOAc. The combined organic layers are washed with water and brine, dried (Na₂SO₄) and concentrated to give a residue, which is purified by column chromatography (SiO₂; CH₂Cl₂/EtOH/NH₃ 90:9:1) to afford the title compound as a colorless solid, m.p. 136-139° C., MS: 415 (M+1)⁺.

### Example 47

### N-{3-{6-[(S)-(Tetrahydro-furan-3-yloxy)-9H-purin-2-ylamino]-phenyl}-methanesulfonamide

This compound can be obtained analogously to Example 23, utilising (S)-(+)-3-hydroxytetrahydrofurane in lieu of cyclopentanol, to afford the title compound as a colorless solid, m.p. 135-138° C., MS: 391 (M+1)⁺

### Example 48

### N-{3-{6-[(R)-(Tetrahydro-furan-3-yloxy)-9H-purin-2-ylamino]-phenyl}-methanesulfonamide

This compound can be obtained analogously to Example 23, utilising (R)-(−)-3-hydroxytetrahydrofurane in lieu of cyclopentanol, to afford the title compound as a colorless solid, m.p. 134-137° C., . MS: 391 (M+1)⁺

### Example 49

### N-{3-[4-((R)-1,2,2-Trimethyl-propylamino)-7H-pyrrolo[2,3-d]pyrimidin-2-ylamino]-phenyl}-methanesulfonamide

This compound can be obtained analogously to Example 1, utilizing (R)-(−)-3,3-dimethyl-2-butylamine in lieu of cyclobutylamine and N-[3-(4-Chloro-7H-pyrrolo[2,3-d]pyrimidin-2-ylamino)-phenyl]-methanesulfonamide (step 42.3) in lieu of N-[3-(6-chloro-9H-purin-2-ylamino)-phenyl]-methanesulfonamide to afford the title compound as a grey solid, m.p. 125-128° C., MS: 403 (M+1)⁺

### Example 50

### N-{3-[6-(2,2-Dimethyl-propylamino)-9H-purin-2-ylamino]-phenyl}-methanesulfonamide

This compound can be obtained analogously to Example 1, utilising neopentylamine in lieu of cyclobutylamine, to afford the title compound as a light pink solid, m.p. 270-273° C., MS: 390 (M+1)⁺.

### Example 51

### N-{3-[4-((S)-1,2,2-Trimethyl-propylamino)-7H-pyrrolo[2,3-d]pyrimidin-2-ylamino]-phenyl}-methanesulfonamide

This compound can be obtained analogously to Example 1, utilizing (S)-(−)-3,3-dimethyl-2-butylamine in lieu of cyclobutylamine and N-[3-(4-Chloro-7H-pyrrolo[2,3-d]pyrimidin-2-ylamino)-phenyl]-methanesulfonamide (step 42.3) in lieu of N-[3-(6-chloro-9H-purin-2-ylamino)-phenyl]-methanesulfonamide to afford the title compound as a grey solid, m.p. 128-132° C., MS: 403 (M+1)⁺.

### Example 52

### N-{3-[6-((R)-1,2-Dimethyl-propylamino)-9H-purin-2-ylamino]-phenyl}-methanesulfonamide

This compound can be obtained analogously to Example 1, utilising (R)-1,2-Dimethyl-propylamine hydrochloride (synthesized according to literature procedure from Moss, N. et al., Synlett (1995), (2), 142 from 3-Methyl-butan-2-one by a four step sequences involving imine formation with R-α-methylbenzylamine followed by a highly diastereoselective reduction with NaBH₄) in lieu of cyclobutylamine, to afford the title compound, as a yellow solid, Rₜ=0.803 min (Acquity UPLC BEH C18, 2.1×50 mm, 1.7 micron, detection 215 nM, 0.1 min 2% CH₃CN in H₂O, 2% to 100% CH₃CN in H₂O in 1.5 min, 0.4 min 100% CH₃CN+0.1% TFA, flow rate 1.0 ml/min); MS: 390 (M+1)⁺.

### Example 53

### N-{3-[6-((S)-1,2-Dimethyl-propylamino)-9H-purin-2-ylamino]-phenyl}-methanesulfonamide

This compound can be obtained analogously to Example 1, utilising (S)-1,2-Dimethyl-propylamine hydrochloride (synthesized analogously to literature procedure from Moss, N. et al., Synlett (1995), (2), 142-4 using S-methylbenzylamine instead of R-α-methylbenzylamine) in lieu of cyclobutylamine, to afford the title compound as a yellow solid, Rₜ=0.804 min (Acquity UPLC BEH C18, 2.1×50 mm, 1.7 micron, detection 215 nM, 0.1 min 2% CH₃CN in H₂O, 2% to 100% CH₃CN in H₂O in 1.5 min, 0.4 min 100% CH₃CN+0.1% TFA, flow rate 1.0 ml/min); MS: 390 (M+1)⁺.

### Example 54

### N-{3-[6-((R)-2,2-Dimethyl-cyclopentylamino)-9H-purin-2-ylamino]-phenyl}-methanesulfonamide

This compound can be obtained analogously to Example 1, utilising (R)-2,2-Dimethyl-cyclopentylamine hydrochloride (synthesized analogously to literature procedure from Moss, N. et al., Synlett (1995), (2), 142 from 2,2-Dimethyl-cyclopentanone) in lieu of cyclobutylamine, to afford the title compound as a pale red solid, Rₜ=0.860 min (Acquity UPLC BEH C18, 2.1×50 mm, 1.7 micron, detection 215 nM, 0.1 min 2% CH₃CN in H₂O, 2% to 100% CH₃CN in H₂O in 1.5 min, 0.4 min 100% CH₃CN+0.1% TFA, flow rate 1.0 ml/min); MS: 416 (M+1)⁺.

### Example 55

### N-{3-[6-((R)-1-Hydroxymethyl-2-methyl-propylamino)-9H-purin-2-ylamino]-phenyl}-methanesulfonamide

This compound can be obtained analogously to Example 1, utilising (R)-2-Amino-3-methyl-butan-1-ol in lieu of cyclobutylamine, to afford the title compound as a beige solid, Rₜ=0.698 min (Acquity UPLC BEH C18, 2.1×50 mm, 1.7 micron, detection 215 nM, 0.1 min 2% CH₃CN in H₂O, 2% to 100% CH₃CN in H₂O in 1.5 min, 0.4 min 100% CH₃CN+0.1% TFA, flow rate 1.0 ml/min); MS: 406 (M+1)⁺.

### Example 56

### N-{3-[6-((S)-1-Hydroxymethyl-2-methyl-propylamino)-9H-purin-2-ylamino]-phenyl}-methanesulfonamide

This compound can be obtained analogously to Example 1, utilising (S)-2-Amino-3-methyl-butan-1-ol in lieu of cyclobutylamine, to afford the title compound as a pale yellow solid, Rₜ=0.696 min (Acquity UPLC BEH C18, 2.1×50 mm, 1.7 micron, detection 215 nM, 0.1 min 2% CH₃CN in H₂O, 2% to 100% CH₃CN in H₂O in 1.5 min, 0.4 min 100% CH₃CN+0.1% TFA, flow rate 1.0 ml/min); MS: 406 (M+1)⁺.

### Example 57

### N-{3-[6-((S)-1-Hydroxymethyl-2,2-dimethyl-propylamino)-9H-purin-2-ylamino]-phenyl}-methanesulfonamide

This compound can be obtained analogously to Example 1, utilising (S)-2-Amino-3,3-dimethyl-butan-1-ol in lieu of cyclobutylamine, to afford the title compound as a pale yellow solid, Rₜ=0.745 min (Acquity UPLC BEH C18, 2.1×50 mm, 1.7 micron, detection 215 nM, 0.1 min 2% CH₃CN in H₂O, 2% to 100% CH₃CN in H₂O in 1.5 min, 0.4 min 100% CH₃CN+0.1% TFA, flow rate 1.0 ml/min); MS: 420 (M+1)⁺.

### Example 58

### N-{3-[6-((R)-1-Hydroxymethyl-2,2-dimethyl-propylamino)-9H-purin-2-ylamino]-phenyl}-methanesulfonamide

This compound can be obtained analogously to Example 1, utilising (R)-2-Amino-3,3-dimethyl-butan-1-ol in lieu of cyclobutylamine, to afford the title compound as a colorless solid, Rₜ=0.744 min (Acquity UPLC BEH C18, 2.1×50 mm, 1.7 micron, detection 215 nM, 0.1 min 2% CH₃CN in H₂O, 2% to 100% CH₃CN in H₂O in 1.5 min, 0.4 min 100% CH₃CN+0.1% TFA, flow rate 1.0 ml/min); MS: 420 (M+1)⁺.

### Example 59

### N-{3-[6-((R)-1-Ethyl-2,2-dimethyl-propylamino)-9H-purin-2-ylamino]-phenyl}-methanesulfonamide

This compound can be obtained analogously to Example 1, utilising (R)-1-Ethyl-2,2-dimethyl-propylamine hydrochloride (synthesized according to literature procedure from Moss, N. et al., Synlett (1995), (2), 142 from 2,2-Dimethyl-pentan-3-one) in lieu of cyclobutylamine, to afford the title compound as a pale pink solid, Rₜ=0.870 min (Acquity UPLC BEH C18, 2.1×50 mm, 1.7 micron, detection 215 nM, 0.1 min 2% CH₃CN in H₂O, 2% to 100% CH₃CN in H₂O in 1.5 min, 0.4 min 100% CH₃CN+0.1% TFA, flow rate 1.0 ml/min); MS: 418 (M+1)⁺.

### Example 60

### N-{3-[4-((R)-2,2-Dimethyl-cyclopentylamino)-7H-pyrrolo[2,3-d]pyrimidin-2-ylamino]-phenyl}-methanesulfonamide

This compound can be obtained analogously to Example 1, utilizing (R)-2,2-Dimethyl-cyclopentylamine hydrochloride in lieu of cyclobutylamine and N-[3-(4-Chloro-7H-pyrrolo[2,3-d]pyrimidin-2-ylamino)-phenyl]-methanesulfonamide (step 42.3) in lieu of N-[3-(6-chloro-9H-purin-2-ylamino)-phenyl]-methanesulfonamide to afford the title compound as a solid, Rₜ=0.958 min (Acquity UPLC BEH C18, 2.1×50 mm, 1.7 micron, detection 215 nM, 0.1 min 2% CH₃CN in H₂O, 2% to 100% CH₃CN in H₂O in 1.5 min, 0.4 min 100% CH₃CN+0.1% TFA, flow rate 1.0 ml/min); MS: 415 (M+1)⁺.

### Example 61

### (S)-2-[2-(3-Methanesulfonylamino-phenylamino)-9H-purin-6-ylamino]-3-methyl-butyramide

This compound can be obtained analogously to Example 1, utilising (S)-2-Amino-3-methyl-butyramide hydrochloride in lieu of cyclobutylamine, to afford the title compound as a red solid, Rₜ=0.652 min (Acquity UPLC BEH C18, 2.1×50 mm, 1.7 micron, detection 215 nM, 0.1 min 2% CH₃CN in H₂O, 2% to 100% CH₃CN in H₂O in 1.5 min, 0.4 min 100% CH₃CN+0.1% TFA, flow rate 1.0 ml/min); MS: 444 (M+1)⁺.

### Example 62

### N-{3-[6-((2S,3aS,6aS)-2-Hydroxymethyl-hexahydro-cyclopenta[b]pyrrol-1-yl)-9H-purin-2-ylamino]-phenyl}-methanesulfonamide

This compound can be obtained analogously to Example 1, utilising (2S,3aS,6aS)-1-(Octahydro-cyclopenta[b]pyrrol-2-yl)-methanol hydrochloride (prepared by reduction of commercial (2S,3aS,6aS)-Octahydro-cyclopenta[b]pyrrole-2-carboxylic acid according to Tetrahedron: Asymmetry 1995, Vol. 6, No. 2, 385-388) in lieu of cyclobutylamine, to afford the title compound as a pale red solid, Rₜ=0.770 min (Acquity UPLC BEH C18, 2.1×50 mm, 1.7 micron, detection 215 nM, 0.1 min 2% CH₃CN in H₂O, 2% to 100% CH₃CN in H₂O in 1.5 min, 0.4 min 100% CH₃CN+0.1% TFA, flow rate 1.0 ml/min); MS: 444 (M+1)⁺.

### Example 63

### N-(3-{6-[(R)-1-(2-Hydroxy-ethyl)-2-methyl-propylamino]-9H-purin-2-ylamino}-phenyl)-methanesulfonamide

This compound can be obtained analogously to Example 1, utilising (R)-3-Amino4-methyl-pentan-1-ol hydrochloride (synthesized analogously to literature procedure from Moss, N. et al., Synlett (1995), (2), 142 from 1-(tert-Butyl-diphenyl-silanyloxy)-4-methyl-pentan-3-one). The latter compound could be prepared by protection of 1-Hydroxy-4-methyl-pentan-3-one prepared according to Synthesis 1990, 1059-1061) in lieu of cyclobutylamine, to afford the title compound as a solid, Rₜ=0.715 min (Acquity UPLC BEH C18, 2.1×50 mm, 1.7 micron, detection 215 nM, 0.1 min 2% CH₃CN in H₂O, 2% to 100% CH₃CN in H₂O in 1.5 min, 0.4 min 100% CH₃CN+0.1% TFA, flow rate 1.0 ml/min); MS: 420 (M+1)⁺.

### Example 64

### N-(3-{[2-(3-Methanesulfonylamino-phenylamino)-9H-purin-6-ylamino]-methyl}-phenyl)-acetamide

This compound can be obtained analogously to Example 1, utilising N-(3-Aminomethyl-phenyl)-acetamide in lieu of cyclobutylamine, to afford the title compound as a beige solid, m.p. 159-162° C., MS: 467 (M+1)⁺

### Example 65

### N-{3-[6-((R)-1,2,2-Trimethyl-butylamino)-9H-purin-2-ylamino]-phenyl}-methanesulfonamide

This compound can be obtained analogously to Example 1, utilising (R)1,2,2-Trimethyl-butylamine hydrochloride (synthesized according to literature procedure from Moss, N. et al., Synlett (1995), (2), 142 from 3,3-Dimethyl-pentan-2-one) in lieu of cyclobutylamine, to afford the title compound as a beige solid, m.p. 143-146° C., MS: 418 (M+1)⁺

### Example 66

### N-(3-{[2-(3-Methanesulfonylamino-phenylamino)-9H-purin-6-ylamino]-methyl}-phenyl)-N-methyl-acetamide

This compound can be obtained analogously to Example 1, utilising N-(3-Aminomethyl-phenyl)-N-methyl-acetamide hydrochloride in lieu of cyclobutylamine, to afford the title compound as a pale pink solid, Rₜ=0.770 min (Acquity UPLC BEH C18, 2.1×50 mm, 1.7 micron, detection 215 nM, 0.1 min 2% CH₃CN in H₂O, 2% to 100% CH₃CN in H₂O in 1.5 min, 0.4 min 100% CH₃CN+0.1% TFA, flow rate 1.0 ml/min); MS: 481 (M+1)⁺

### Example 67

### N-{3-[6-(3-Trifluoromethyl-benzylamino)-9H-purin-2-ylamino]-phenyl}-methanesulfonamide

This compound can be obtained analogously to Example 1, utilising 3-Trifluoromethyl-benzylamine in lieu of cyclobutylamine, to afford the title compound as a pale red foam, Rₜ=0.902 min (Acquity UPLC BEH C18, 2.1×50 mm, 1.7 micron, detection 215 nM, 0.1 min 2% CH₃CN in H₂O, 2% to 100% CH₃CN in H₂O in 1.5 min, 0.4 min 100% CH₃CN+0.1% TFA, flow rate 1.0 ml/min); MS: 478 (M+1)⁺.

### Example 68

### N-{3-[6-(3-Trifluoromethoxy-benzylamino)-9H-purin-2-ylamino]-phenyl}-methanesulfonamide

This compound can be obtained analogously to Example 1, utilising 3-Trifluoromethoxy-benzylamine in lieu of cyclobutylamine, to afford the title compound as a pale red solid, Rₜ=0.925 min (Acquity UPLC BEH C18, 2.1×50 mm, 1.7 micron, detection 215 nM, 0.1 min 2% CH₃CN in H₂O, 2% to 100% CH₃CN in H₂O in 1.5 min, 0.4 min 100% CH₃CN+0.1% TFA, flow rate 1.0 ml/min); MS: 494 (M+1)⁺.

### Example 69

### N-{3-[6-(3-Methoxy-benzylamino)-9H-purin-2-ylamino]-phenyl}-methanesulfonamide

This compound can be obtained analogously to Example 1, utilising 3-Methoxy-benzylamine in lieu of cyclobutylamine, to afford the title compound as a pale red solid, Rₜ=0.808 min (Acquity UPLC BEH C18, 2.1×50 mm, 1.7 micron, detection 215 nM, 0.1 min 2% CH₃CN in H₂O, 2% to 100% CH₃CN in H₂O in 1.5 min, 0.4 min 100% CH₃CN+0.1% TFA, flow rate 1.0 ml/min); MS: 440 (M+1)⁺

### Example 70

### N-{3-[6-(3-Nitro-benzylamino)-9H-purin-2-ylamino]-phenyl}-methanesulfonamide

This compound can be obtained analogously to Example 1, utilising 3-Nitro-benzylamine in lieu of cyclobutylamine, to afford the title compound as an orange foam, Rₜ=0.801 min (Acquity UPLC BEH C18, 2.1×50 mm, 1.7 micron, detection 215 nM, 0.1 min 2% CH₃CN in H₂O, 2% to 100% CH₃CN in H₂O in 1.5 min, 0.4 min 100% CH₃CN+0.1% TFA, flow rate 1.0 ml/min); MS: 455 (M+1)⁺.

### Example 71

### N-{3-[6-((1S,2R-2-Hydroxy-cyclopentylamino)-9H-purin-2-ylamino]-phenyl}-methanesulfonamide

This compound can be obtained analogously to Example 1, utilising (1 R,2S)-2-Amino-cyclopentanol hydrochloride in lieu of cyclobutylamine, to afford the title compound as pale brown solid, Rₜ=0.678 min (Acquity UPLC BEH C18, 2.1×50 mm, 1.7 micron, detection 215 nM, 0.1 min 2% CH₃CN in H₂O, 2% to 100% CH₃CN in H₂O in 1.5 min, 0.4 min 100% CH₃CN+0.1% TFA, flow rate 1.0 ml/min); MS: 404 (M+1)⁺

### Example 72

### N-{3-[6-(3-Amino-benzylamino)-9H-purin-2-ylamino]-phenyl}-methanesulfonamide

This compound can be obtained analogously to Example 1, utilising 3-Aminomethyl-phenylamine in lieu of cyclobutylamine, to afford the title compound as a beige solid, m.p. 170-173° C., MS: 425 (M+1)⁺

### Example 73

### N-{3-[6-(3-Hydroxy-benzylamino)-9H-purin-2-ylamino]-phenyl}-methanesulfonamide

This compound can be obtained analogously to Example 1, utilising 3-Aminomethyl-phenol in lieu of cyclobutylamine, to afford the title compound as a beige solid, m.p. 221-225° C., MS: 426 (M+1)⁺

### Example 74

### N-[3-(6-Benzylamino-9H-purin-2-ylamino)-phenyl]-methanesulfonamide

This compound can be obtained analogously to Example 1, utilising benzylamine in lieu of cyclobutylamine, to afford the title compound as a red foam, Rₜ=0.804 min (Acquity UPLC BEH C18, 2.1×50 mm, 1.7 micron, detection 215 nM, 0.1 min 2% CH₃CN in H₂O, 2% to 100% CH₃CN in H₂O in 1.5 min, 0.4 min 100% CH₃CN+0.1% TFA, flow rate 1.0 ml/min); MS: 410 (M+1)⁺

### Example 75

### N-{3-[6-(3-Methanesulfonyl-benzylamino)-9H-purin-2-ylamino]-phenyl}-methanesulfonamide

This compound can be obtained analogously to Example 1, utilising 3-Methanesulfonyl-benzylamine in lieu of cyclobutylamine, to afford the title compound as a red solid, Rₜ=0.703 min (Acquity UPLC BEH C18, 2.1×50 mm, 1.7 micron, detection 215 nM, 0.1 min 2% CH₃CN in H₂O, 2% to 100% CH₃CN in H₂O in 1.5 min, 0.4 min 100% CH₃CN+0.1% TFA, flow rate 1.0 ml/min); MS: 488 (M+1 )⁺

### Example 76

### N-{3-[6-((1S,2S)-2-Hydroxy-cyclopentylamino)-9H-purin-2-ylamino]-phenyl}-methanesulfonamide

This compound can be obtained analogously to Example 1, utilising (1S,2S)-2-Amino-cyclopentanol hydrochloride in lieu of cyclobutylamine, to afford the title compound as pale red foam, Rₜ=0.675 min (Acquity UPLC BEH C18, 2.1×50 mm, 1.7 micron, detection 215 nM, 0.1 min 2% CH₃CN in H₂O, 2% to 100% CH₃CN in H₂O in 1.5 min, 0.4 min 100% CH₃CN+0.1% TFA, flow rate 1.0 ml/min); MS: 404 (M+1)⁺

### Example 77

### N-(3-{6-[3-(4-Methyl-piperazin-1-yl)-benzylamino]-9H-purin-2-ylamino}-phenyl)-methanesulfonamide

This compound can be obtained analogously to Example 1, utilising 3-(4-Methyl-piperazin-1-yl)-benzylamine in lieu of cyclobutylamine, to afford the title compound as a brown solid, Rₜ=0.646 min (Acquity UPLC BEH C18, 2.1×50 mm, 1.7 micron, detection 215 nM, 0.1 min 2% CH₃CN in H₂O, 2% to 100% CH₃CN in H₂O in 1.5 min, 0.4 min 100% CH₃CN+0.1% TFA, flow rate 1.0 ml/min); MS: 508 (M+1)⁺

### Example 78

### N-{3-[6-(3-Cyano-benzylamino)-9H-purin-2-ylamino]-phenyl}-methanesulfonamide

This compound can be obtained analogously to Example 1, utilising 3-Aminomethyl-benzonitrile in lieu of cyclobutylamine, to afford the title compound as a pink solid, m.p. 128-131° C., MS: 435 (M+1)⁺

### Example 79

### N-{3-[4-((2S,3aS,6aS)-2-Hydroxymethyl-hexahydro-cyclopenta[b]pyrrol-1-yl)-7H-pyrrolo[2,3-d]pyrimidin-2-ylamino]-phenyl}-methanesulfonamide

This compound can be obtained analogously to Example 1, utilizing ((2S,3aS,6aS)-1-(Octahydro-cyclopenta[b]pyrrol-2-yl)-methanol hydrochloride in lieu of cyclobutylamine and N-[3-(4-Chloro-7H-pyrrolo[2,3-d]pyrimidin-2-ylamino)-phenyl]-methanesulfonamide (step 42.3) in lieu of N-[3-(6-chloro-9H-purin-2-ylamino)-phenyl]-methanesulfonamide to afford the title compound as a solid, Rₜ=0.864 min (Acquity UPLC BEH C18, 2.1×50 mm, 1.7 micron, detection 215 nM, 0.1 min 2% CH₃CN in H₂O, 2% to 100% CH₃CN in H₂O in 1.5 min, 0.4 min 100% CH₃CN+0.1% TFA, flow rate 1.0 ml/min); MS: 443 (M+1)⁺

### Example 80

### N-{3-[6-(1,4-Diaza-spiro[5.5]undec-1-yl)-9H-purin-2-ylamino]-phenyl}-methanesulfonamide

This compound can be obtained analogously to Example 1, utilising 1,4-Diaza-spiro[5.5]undecane4-carboxylic acid tert-butyl ester (prepared by BOC protection of 1,4-Diaza-spiro[5.5]undecane) in lieu of cyclobutylamine, to afford the title compound as a solid, Rₜ=0.646 min (Acquity UPLC BEH C18, 2.1×50 mm, 1.7 micron, detection 215 nM, 0.1 min 2% CH₃CN in H₂O, 2% to 100% CH₃CN in H₂O in 1.5 min, 0.4 min 100% CH₃CN+0.1% TFA, flow rate 1.0 ml/min); MS: 457 (M+1 )⁺

### Example 81

### N-{3-[6-(1,4-Diaza-spiro[5.5]undec-4-yl)-9H-purin-2-ylamino]-phenyl}-methanesulfonamide

This compound can be obtained analogously to Example 1, utilising 1,4-Diaza-spiro[5.5]undecane in lieu of cyclobutylamine, to afford the title compound as a colorless solid, Rₜ=0.644 min (Acquity UPLC BEH C18, 2.1×50 mm, 1.7 micron, detection 215 nM, 0.1 min 2% CH₃CN in H₂O, 2% to 100% CH₃CN in H₂O in 1.5 min, 0.4 min 100% CH₃CN+0.1% TFA, flow rate 1.0 ml/min); MS: 457 (M+1)⁺

### Example 82

### N-{3-[4-(2-Isopropylamino-ethylamino)-7H-pyrrolo[2,3-d]pyrimidin-2-ylamino]-phenyl}-methanesulfonamide

This compound can be obtained analogously to Example 1, utilising N*1*-Isopropyl-ethane-1,2-diamine in lieu of cyclobutylamine, and N-[3-(4-Chloro-7H-pyrrolo[2,3-d]pyrimidin-2-ylamino)-phenyl]-methanesulfonamide (step 42.3) in lieu of N-[3-(6-chloro-9H-purin-2-ylamino)-phenyl]-methanesulfonamide to afford the title compound as a yellow solid, m.p. 127-131° C., MS: 404 (M+1)⁺

### Example 83

### N-[3-(4-Cyclopropylamino-7H-pyrrolo[2,3-d]pyrimidin-2-ylamino)-phenyl]-methanesulfonamide

This compound can be obtained analogously to Example 1, utilising cyclopropylamine in lieu of cyclobutylamine, and N-[3-(4-Chloro-7H-pyrrolo[2,3-d]pyrimidin-2-ylamino)-phenyl]-methanesulfonamide (step 42.3) in lieu of N-[3-(6-chloro-9H-purin-2-ylamino)-phenyl]-methanesulfonamide to afford the title compound as a pale yellow foam, Rₜ=0.792 min (Acquity UPLC BEH C18, 2.1×50 mm, 1.7 micron, detection 215 nM, 0.1 min 2% CH₃CN in H₂O, 2% to 100% CH₃CN in H₂O in 1.5 min, 0.4 min 100% CH₃CN+0.1% TFA, flow rate 1.0 ml/min); MS: 359 (M+1)⁺.

### Example 84

### N-{3-[4-((S)-1-Hydroxymethyl-2-methyl-propylamino)-7H-pyrrolo[2,3-d]pyrimidin-2-ylamino]-phenyl}-methanesulfonamide

This compound can be obtained analogously to Example 1, utilising (S)-2-Amino-3-methyl-butan-1-ol in lieu of cyclobutylamine, and N-[3-(4-Chloro-7H-pyrrolo[2,3-d]pyrimidin-2-ylamino)-phenyl]-methanesulfonamide (step 42.3) in lieu of N-[3-(6-chloro-9H-purin-2-ylamino)-phenyl]-methanesulfonamide to afford the title compound as a colorless solid, m.p. 106-109° C., MS: 405 (M+1)⁺

### Example 85

### N-[3-(4-Isopropylamino-7H-pyrrolo[2,3-d]pyrimidin-2-ylamino)-phenyl]-methanesulfonamide

This compound can be obtained analogously to Example 1, utilising isopropylamine in lieu of cyclobutylamine, and N-[3-(4-Chloro-7H-pyrrolo[2,3-d]pyrimidin-2-ylamino)-phenyl]-methanesulfonamide (step 42.3) in lieu of N-[3-(6-chloro-9H-purin-2-ylamino)-phenyl]-methanesulfonamide to afford the title compound as a grey foam, Rₜ=0.803 min (Acquity UPLC BEH C18, 2.1×50 mm, 1.7 micron, detection 215 nM, 0.1 min 2% CH₃CN in H₂O, 2% to 100% CH₃CN in H₂O in 1.5 min, 0.4 min 100% CH₃CN+0.1% TFA, flow rate 1.0 ml/min); MS: 361 (M+1)⁺

### Example 86

### N-[3-(4-Cyclobutylamino-7H-pyrrolo[2,3-d]pyrimidin-2-ylamino)-phenyl]-methanesulfonamide

This compound can be obtained analogously to Example 1, utilising N-[3-(4-Chloro-7H-pyrrolo[2,3-d]pyrimidin-2-ylamino)-phenyl]-methanesulfonamide (step 42.3) in lieu of N-[3-(6-chloro-9H-purin-2-ylamino)-phenyl]-methanesulfonamide to afford the title compound as a colorless foam, Rₜ=0.835 min (Acquity UPLC BEH C18, 2.1×50 mm, 1.7 micron, detection 215 nM, 0.1 min 2% CH₃CN in H₂O, 2% to 100% CH₃CN in H₂O in 1.5 min, 0.4 min 100% CH₃CN+0.1% TFA, flow rate 1.0 ml/min); MS: 373 (M+1)⁺.

### Example 87

### N-(3-{4-[(2-Hydroxy-ethyl)-isopropyl-amino]-7H-pyrrolo[2,3-d]pyrimidin-2-ylamino}-phenyl)-methanesulfonamide

This compound can be obtained analogously to Example 1, utilising 2-Isopropylamino-ethanol in lieu of cyclobutylamine, and N-[3-(4-Chloro-7H-pyrrolo[2,3-d]pyrimidin-2-ylamino)-phenyl]-methanesulfonamide (step 42.3) in lieu of N-[3-(6-chloro-9H-purin-2-ylamino)-phenyl]-methanesulfonamide to afford the title compound as a beige solid, m.p. 129-132° C., MS: 405 (M+1)⁺

### Example 88

### N-{3-[6-(3-Acetyl-benzylamino)-9H-purin-2-ylamino]-phenyl}-methanesulfonamide

This compound can be obtained analogously to Example 1, utilising 3-(2-Methyl-[1,3]dioxolan-2-yl)-benzylamine (prepared according to Journal of Medicinal Chemistry (2000), 43(17), 3315-3321) in lieu of cyclobutylamine, to afford, after acidic hydrolysis of the acetal, the title compound as a colorless solid, Rₜ=0.762 min (Acquity UPLC BEH C18, 2.1×50 mm, 1.7 micron, detection 215 nM, 0.1 min 2% CH₃CN in H₂O, 2% to 100% CH₃CN in H₂O in 1.5 min, 0.4 min 100% CH₃CN+0.1% TFA, flow rate 1.0 ml/min); MS: 452 (M+1)⁺

### Example 89

### N-(3-{6-[(Benzo[1,2,5]oxadiazol-5-ylmethyl)-amino]-9H-purin-2-ylamino}-phenyl)-methanesulfonamide

This compound can be obtained analogously to Example 1, utilising C-Benzo[1,2,5]oxadiazol-5-yl-methylamine (prepared according to WO2001005783 from 5-bromomethylbenzofuroxane) in lieu of cyclobutylamine, to afford the title compound as a pale orange solid, Rₜ=0.796 min (Acquity UPLC BEH C18, 2.1×50 mm, 1.7 micron, detection 215 nM, 0.1 min 2% CH₃CN in H₂O, 2% to 100% CH₃CN in H₂O in 1.5 min, 0.4 min 100% CH₃CN+0.1% TFA, flow rate 1.0 ml/min); MS: 452 (M+1)⁺

### Example 90

### N-(3-{6-[2-(2-Hydroxy-ethyl)-benzylamino]-9H-purin-2-ylamino}-phenyl)-methanesulfonamide

This compound can be obtained analogously to Example 1, utilising 2-(2-Aminomethyl-phenyl)-ethanol in lieu of cyclobutylamine, to afford the title compound as a pale pink solid, Rₜ=0.740 min (Acquity UPLC BEH C18, 2.1×50 mm, 1.7 micron, detection 215 nM, 0.1 min 2% CH₃CN in H₂O, 2% to 100% CH₃CN in H₂O in 1.5 min, 0.4 min 100% CH₃CN+0.1% TFA, flow rate 1.0 ml/min); MS: 454 (M+1)⁺.

### Example 91

### N-{3-[4-((R)-1,2,2-Trimethyl-butylamino)-7H-pyrrolo[2,3-d]pyrimidin-2-ylamino]-phenyl}-methanesulfonamide

This compound can be obtained analogously to Example 1, utilizing (R)-1,2,2-Trimethyl-butylamine hydrochloride in lieu of cyclobutylamine, N-[3-(4-Chloro-7H-pyrrolo[2,3-d]pyrimidin-2-ylamino)-phenyl]-methanesulfonamide (step 42.3) in lieu of N-[3-(6-chloro-9H-purin-2-ylamino)-phenyl]-methanesulfonamide to afford the title compound as a yellow solid, Rₜ=1.002 min (Acquity UPLC BEH C18, 2.1×50 mm, 1.7 micron, detection 215 nM, 0.1 min 2% CH₃CN in H₂O, 2% to 100% CH₃CN in H₂O in 1.5 min, 0.4 min 100% CH₃CN+0.1% TFA, flow rate 1.0 ml/min); MS: 417 (M+1)⁺

### Example 92

### N-{3-[6-((S)-1-Phenyl-propylamino)-9H-purin-2-ylamino]-phenyl}-methanesulfonamide

This compound can be obtained analogously to Example 1, utilising (S)-1-Phenyl-propylamine in lieu of cyclobutylamine, to afford the title compound as a pale pink solid, m.p. 138-141° C., MS: 438 (M+1)⁺

### Example 93

### N-{3-[6-(2-Hydroxymethyl-benzylamino)-9H-purin-2-ylamino]-phenyl}-methanesulfonamide

This compound can be obtained analogously to Example 1, utilising (2-Aminomethyl-phenyl)-methanol in lieu of cyclobutylamine, to afford the title compound as a pink solid, m.p. 145-148° C., MS: 440 (M+1)⁺

### Example 94

### N-{3-[6-(2-Hydroxy-benzylamino)-9H-purin-2-ylamino]-phenyl}-methanesulfonamide

This compound can be obtained analogously to Example 1, utilising 2-Aminomethyl-phenol in lieu of cyclobutylamine, to afford the title compound as a pink solid, m.p. 168-171° C., MS: 426 (M+1)⁺

### Example 95

### N-(3-{6-[(2-Amino-ethyl)-isopropyl-amino]-9H-purin-2-ylamino}-phenyl)-methanesulfonamide

This compound can be obtained analogously to Example 1, utilising 2-(2-isopropylamino-ethyl)-isoindole-1,3-dione in lieu of cyclobutylamine, to afford, after deprotection of the phtalimide with hydrazine, the title compound as a pale beige solid, Rₜ=0.594 min (Acquity UPLC BEH C18, 2.1×50 mm, 1.7 micron, detection 215 nM, 0.1 min 2% CH₃CN in H₂O, 2% to 100% CH₃CN in H₂O in 1.5 min, 0.4 min 100% CH₃CN+0.1% TFA, flow rate 1.0 ml/min); MS: 405 (M+1)⁺

### Example 96

### N-(3-{4-[2-(2-Hydroxy-ethyl)-benzylamino]-7H-pyrrolo[2,3-d]pyrimidin-2-ylamino}-phenyl)-methanesulfonamide

This compound can be obtained analogously to Example 1, utilizing 2-(2-Aminomethyl-phenyl)-ethanol in lieu of cyclobutylamine, N-[3-(4-Chloro-7H-pyrrolo[2,3-d]pyrimidin-2-ylamino)-phenyl]-methanesulfonamide (step 42.3) in lieu of N-[3-(6-chloro-9H-purin-2-ylamino)-phenyl]-methanesulfonamide to afford the title compound as a pale brown foam, Rₜ=0.808 min (Acquity UPLC BEH C18, 2.1×50 mm, 1.7 micron, detection 215 nM, 0.1 min 2% CH₃CN in H₂O, 2% to 100% CH₃CN in H₂O in 1.5 min, 0.4 min 100% CH₃CN+0.1% TFA, flow rate 1.0 ml/min); MS: 453 (M+1)⁺

### Example 97

### Ethenesulfonic acid {3-[6-((R)-1,2,2-trimethyl-propylamino)-9H-purin-2-ylamino]-phenyl}-amide

HCl (2M in H2O, 10 mL) is added to a solution of ethenesulfonic acid {3-[9-(tetrahydro-pyran-2-yl)-6-((R)-1,2,2-trimethyl-propylamino)-9H-purin-2-ylamino]-phenyl}-amide (step 97.3, 58 mg, 0.116 mmol) in MeOH (10 mL). After 1 h at room temperature, the mixture is neutralized with NaOH (2M, 10 mL). The solvent is evaporated off and the residue is dissolved in EtOAc, and NaHCO₃ is then added. The aqueous phase is separated and extracted with EtOAc. The combined organic layers are washed with water and brine, dried (Na₂SO₄) and concentrated to give a residue, which is purified by reversed phase MPLC (Buchi system), yielding, after neutralization with saturated aqueous NaHCO3, the title compound as a colorless solid, m.p. 133-136° C., MS: 416 (M+1)⁺

The starting material is prepared as follows:

Step 97.1: [2-Chloro-9-(tetrahydro-pyran-2-yl)-9H-purin-6-yl]-((R)-1,2,2-trimethyl-propyl)-amine

This compound can be obtained analogously to Example 1, utilizing (R)-(−)-3,3-dimethyl-2-butylamine in lieu of cyclobutylamine and 2,6-Dichloro-9-(tetrahydro-pyran-2-yl)-9H-purine in lieu of N-[3-(6-chloro-9H-purin-2-ylamino)-phenyl]-methanesulfonamide to afford the title compound as a colorless foam, MS: 338 (M+1)⁺.

Step 97.2: 2-Benzyloxy-ethanesulfonic acid (3-amino-phenyl)-amide

This compound can be obtained analogously to example 27.1 and 27.2 utilising 2-Benzyloxy-ethanesulfonyl chloride (prepared as described WO2006033446) in lieu of trifluoromethanesulfonic anhydride, to afford the title compound as brown oil, MS: 335 (M−1)⁻.

Step 97.3: Ethenesulfonic acid {3-[9-(tetrahydro-pyran-2-yl)-6-((R)-1,2,2-trimethyl-propylamino)-9H-purin-2-ylamino]-phenyl}-amide

A mixture of [2-Chloro-9-(tetrahydro-pyran-2-yl)-9H-purin-6-yl]-((R)-1,2,2-trimethyl-propyl)-amine (step 97.1, 177 mg, 0.523 mmol), 2-Benzyloxy-ethanesulfonic acid (3-amino-phenyl)-amide (step 97.2; 200 mg, 0.653 mmol), (R)-(+)-2,2′-Bis(diphenylphosphino)-1,1′-binaphthalene (16.3 mg, 0.0262 mmol), palladium (II) acetate (6 mg, 0.0262 mmol), cesium carbonate (190 mg, 0.576 mmol) in dry dimethylacetamide (5 mL) is heated under an argon atmosphere at 150° C. for 5 h. The cooled suspension is diluted with water, filtered (hyflo) and the residue is dissolved in EtOAc and water is added. The aqueous phase is separated and extracted with EtOAc. The combined organic layers are washed with water and brine, dried (Na₂SO₄) and concentrated to give a residue, which is purified by column chromatography (SiO₂; CH₂Cl₂/EtOAc 2:1 to 1:1) to afford the title compound as a yellow solid, MS: 500 (M+1)⁺.

### Example 98

### Soft Capsules

5000 soft gelatin capsules, each comprising as active ingredient 0.05 g of one of the compounds of formula I mentioned in the preceding Examples, are prepared as follows:

### Composition

| | | |
|---|---|---|
| Active ingredient | 250 | g |
| Lauroglycol | 2 | litres |

Preparation process: The pulverized active ingredient is suspended in Lauroglykol® (propylene glycol laurate, Gattefosse S. A., Saint Priest, France) and ground in a wet pulverizer to produce a particle size of about 1 to 3 μm. 0.419 g portions of the mixture are then introduced into soft gelatin capsules using a capsule-filling machine.

1. A sulphonamidoaniline of formula I, 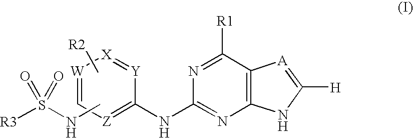
wherein
A is N or CH,
W, X, Y and Z are N or CH under the proviso that at least one of the three symbols W, X and Y represent CH,
R¹ represents NR₄R₅ or OR₄, wherein
R₄ represents optionally substituted alkyl, optionally substituted cycloalkyl optionally comprising one or two nitrogen or oxygen atoms, or substituted aryl, and
R₅ represents hydrogen or unsubstituted or substituted alkyl, or
R₄ and R₅ together with the nitrogen to which they are attached represent an optionally substituted five- or six-membered nitrogen containing monocyclic ring, an optionally substituted nitrogen containing fully saturated bicyclic ring, or an spirocyclic fully saturated ring system containing one or two nitrogen atoms,
R² is hydrogen, lower alkenyl or alkyl,
R³ is alkyl which is unsubstituted or mono-, di- or trisubstituted by halogen; alkenyl or aryl, or a salt thereof.

2. The sulphonamidoaniline of formula I according to claim 1, wherein
A is N or CH,
W, X, Y and Z are N or CH under the proviso that at least one of the three symbols W, X and Y represent CH,
R₁ represents NR₄R₅ or OR₄, wherein
R₄ is selected from
alkyl which is unsubstituted or substituted by hydroxy, lower alkoxy, amido, phenyl, amino-phenyl, di-(lower alkyl)amino-phenyl, trifluoromethyl phenyl, trifluoromethoxy phenyl, cyano phenyl, cyano lower alkyl phenyl, lower alkanoyl phenyl, lower alkanoyl amino-phenyl, lower alkanoyl (lower alkyl) amino-phenyl, lower alkyl sulfonylamino phenyl, lower alkoxy phenyl, hydroxy phenyl, hydroxy lower alkyl phenyl, 4-lower alkyl-piperazin-1-yl)-phenyl, nitro phenyl, imidazolyl, morpholinyl, di-(lower alkyl)amino, cyano, N-lower alkyl amino, C₅-C₇-cycloalkyl, benzo[1,2,5]oxadiazolyl, pyridyl or piperidinyl, or 1-aza-bicyclo[2.2.2]octyl, tetrahydrofuranyl, C₃-C₅-cycloalkyl being unsubstituted or substituted by lower alkyl or hydroxy; phenyl which is substituted by halogen; and
R₅ represents hydrogen or lower ally being unsubstituted or substituted by hydroxyl or amino, or
R₄ and R₅ together with the nitrogen atom to which they are attached represent morpholinyl, pyrrolidinyl which is unsubstituted or substituted by hydroxy lower alkyl or hydroxyl; piperazinyl substituted by pyridyl or lower alkyl; hexahydro-cyclopenta[b]pyrrol-1-yl which is unsubstituted or substituted by hydroxy lower alkyl; or diaza-spiro[5.5]undecyl,
R₂ is hydrogen, and
R₃ is lower alkyl, which is unsubstituted or mono-, di or trisubstituted by halogen, lower alkenyl, or phenyl monosubstituted by halogen,
or a salt thereof.

3. The sulphonamidoaniline of formula I according to claim 1, wherein
A is N,
W, X, Y and Z are all CH,
R₁ represents NR₄R₅ or OR₄, wherein
R₄ represents isopropyl, 1,2,2-trimethyl-propyl, 2,2-dimethyl-propyl, 1,2-dimethyl-propyl, 1-ethyl-2,2-dimethyl-propyl, 2-hydroxy-1,1-dimethyl-ethyl, 1,2,2-trimethyl-butyl, 2-hydroxy-ethyl, 1-hydroxymethyl-2-methyl-propyl, 1-(2-hydroxy-ethyl)-2-methyl-propyl, 1-hydroxymethyl-2,2-dimethyl-propyl, 2-methoxy-ethyl, 2-isopropylamino-ethyl, 3-methyl-butyramide, benzyl, amino-benzyl, 3-dimethylamino-benzyl, 4-dimethylamino-benzyl, 3-acetylamino-benzyl, 3-(acetyl)-N-methylamino-benzyl, 3-cyano-benzyl, 4-cyanomethyl-benzyl, 3-acetyl-benzyl, methanesulfonylamino-benzyl, 3-(4methyl-piperazin-1-yl)-benzyl, 3-trifluoromethyl-benzyl, 3-trifluoromethoxy-benzyl, 3-hydroxy-benzyl, 2-hydroxymethyl-benzyl, 2-hydroxyethyl-benzyl, 3-methoxy-benzyl, 3-nitro-benzyl, benzo[1,2,5]oxadiazol-5-ylmethyl, 1-phenyl-ethyl, 1-phenyl-propyl, 4-imidazolylethyl, 1H-Imidazol-2-ylmethyl, morpholin-4-yl-ethyl, diisopropylaminoethyl, dimethylaminoethyl, cyanoethyl, 2,3-dihydroxy-propyl, cyclohexylmethyl, 2-pyridin-2-yl-ethyl, pyridin-3-ylmethyl, piperidin-2-ylmethyl, 1-aza-bicyclo[2.2.2]oct-3-yl, tetrahydro-furan3-yl, cyclopropyl, cyclobutyl, cyclopentyl, dimethyl-cyclopentyl, 2-hydroxy-cyclopentyl, 4-fluoro-phenyl, and
R₅ represents hydrogen, methyl, 2-amino-ethyl or 2-hydroxy-ethyl, or
R₄ and R₅ together with the nitrogen atom to which they are attached represent 2-hydroxymethyl-hexahydro-cyclopenta[b]pyrrol-1-yl, 1,4-diaza-spiro[5.5]undec-1-yl, 1,4-diaza-spiro[5.5]undec-4-yl, pyrrolidinyl which is unsubstituted or substituted by hydroxymethyl or hydroxy, piperazinyl substituted by 4-pyridyl or methyl, 4-methyl-imidazol-1-yl, morpholin-4-yl,
R₂ is hydrogen, and
R₃ is lower alkyl, which is unsubstituted or mono-, di or trisubstituted by fluoro, vinyl, or phenyl monosubstituted by fluoro,
or a salt thereof.

4. A sulphonamidoaniline of formula I according to claim 1, wherein
A is N or CH,
W, X, Y and Z are N or CH under the proviso that at least one of the three symbols W, X and Y represent CH,
R¹ represents NR₄R₅ or OR₄, wherein
R₄ represents optionally substituted alkyl, cycloalkyl optionally comprising one or two nitrogen or oxygen atoms or bicycloalkyl, and
R₅ represents hydrogen or alkyl, or
R₄ and R₅ together with the nitrogen to which they are attached represent an optionally substituted five- or six-membered nitrogen containing ring,
R² is hydrogen or alkyl,
R³is alkyl, haloalkyl or haloaryl,
or a salt thereof.

5. The sulphonamidoaniline of formula I according to claim 4, wherein
A is N or CH,
W, X, Y and Z are N or CH under the proviso that at least one of the three symbols W, X and Y represent CH,
R₁ represents NR₄R₅ or OR₄, wherein
R₄ is selected from
alkyl which is unsubstituted or substituted by hydroxy, lower alkoxy, phenyl, di-(lower alkyl)amino-phenyl, cyano lower alkyl phenyl, lower alkyl sulfonylamino phenyl, imidazolyl, morpholinyl, di-(lower alkyl)amino, cyano, C₅-C₇-cycloalkyl, pyridyl or piperidinyl, or
1-aza-bicyclo[2.2.2]octyl, tetrahydrofuranyl, cyclobutyl or cyclopentyl; and
R₅ represents hydrogen or lower alkyl, or
R₄ and R₅ together with the nitrogen atom to which they are attached represent morpholinyl, pyrrolidinyl which is unsubstituted or substituted by hydroxy lower alkyl or hydroxy, or piperazinyl substituted by pyridyl or lower alkyl,
R₂ is hydrogen, and
R₃ is lower alkyl, which is unsubstituted or mono-, di or trisubstituted by halogen, or phenyl monosubstituted by halogen,
or a salt thereof.

6. The sulphonamidoaniline of formula I according to claim 4, wherein
A is N,
W, X, Y and Z are all CH,
R₁ represents NR₄R₅ or OR₄, wherein
R₄ represents isopropyl, 1,2,2-trimethyl-propyl, 2-hydroxy-1,1-dimethyl-ethyl, 2-hydroxy-ethyl, 2-methoxy-ethyl, benzyl, 3dimethylamino-benzyl, 4-dimethylamino-benzyl, 4-cyanomethyl-benzyl, 4-methanesulfonylamino-benzyl, 1-phenyl-ethyl, 4-imidazolylethyl, 2-morpholin-4-yl-ethyl, diisopropylaminoethyl, dimethylaminoethyl, cyanoethyl, 2,3-dihydroxy-propyl, cyclohexylmethyl, 2-pyridin-2-yl-ethyl, pyridin-3-ylmethyl, piperidin-2-ylmethyl, 1-aza-bicyclo[2.2.2]oct-3-yl, tetrahydro-furan-3-yl, cyclobutyl, cyclopentyl, and
R₅ represents hydrogen or methyl, or
R₄ and R₅ together with the nitrogen atom to which they are attached represent pyrrolidinyl which is unsubstituted or substituted by hydroxymethyl or hydroxy, piperazinyl substituted by 4-pyridyl or methyl, 4-methyl-imidazol-1-yl, morpholin-4-yl,
R₂ is hydrogen, and
R₃ is lower alkyl, which is unsubstituted or mono-, di or trisubstituted by fluoro, or phenyl monosubstituted by fluoro,
or a salt thereof.

7. Use of a sulphonamidoaniline of formula I, 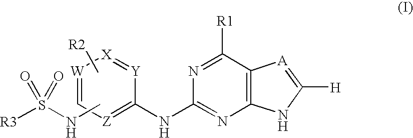
according to claim 1, or a pharmaceutically acceptable salt of such a compound, for the preparation of a pharmaceutical product for the treatment of a proliferative disease.

8. A method for the treatment of a proliferative disease which responds to an inhibition of the JAK-2-receptor tyrosine kinase activity, which comprises administering a sulphonamidoaniline of formula I or a pharmaceutically acceptable salt of such sulphonamidoaniline, wherein the radicals and symbols have the meanings as defined in claim 1, in a quantity effective against the said disease, to a warm-blooded animal requiring such treatment.

9. A pharmaceutical preparation comprising a sulphonamidoaniline of formula I according to claim 1, or a pharmaceutically acceptable salt of such a compound, and at least one pharmaceutically acceptable carrier.
